(19) United States
(12) Patent Application Publication
(10) Pub. No.: US 2020/0401222 A1
(43) Pub. Date: Dec. 24, 2020

(54) Gaming Cognitive Performance

(71) Applicant: MaddCog Limited, Wanaka (NZ)

(72) Inventors: Ben Wisbey, Wanaka (NZ); Guillaume Mathias, Midway, UT (US)

(73) Assignee: MaddCog Limited, Wanaka (NZ)

(21) Appl. No.: 16/869,187

(22) Filed: May 7, 2020

Related U.S. Application Data
(60) Provisional application No. 62/863,662, filed on Jun. 19, 2019.
(60) Provisional application No. 62/863,685, filed on Jun. 19, 2019.
(60) Provisional application No. 62/863,697, filed on Jun. 19, 2019.

(51) Int. Cl. G06F 3/01; A61B 5/16; A61B 5/024; A61B 5/00; G06N 7/00; H04R 5/033

(52) U.S. Cl. CPC G06F 3/015; A61B 5/165; A61B 5/02405; H04R 5/033; A61B 5/6803; G06N 7/005; A61B 5/746

(57) ABSTRACT

Apparatus and associated methods relate to capturing physiological data from a sensor configured in a user's wearable device while the user performs a task, individualizing the physiological data to the user based on comparison with historical user physiological data, measuring the user's cognitive function determined in relationship to the individual's physiological data, and automatically notifying the user of cognitive fatigue and performance detected based on evaluating the measured cognitive function over time. In an illustrative example, the wearable device maybe a gaming headset. The measured cognitive function may be, for example, determined as a function of electroencephalograph or heart rate variability data captured from a user while the user performs a task. Some examples may provide recovery recommendations based on the detected cognitive fatigue. Various embodiments may advantageously recommend a recovery schedule determined as a function of a user's historical physiological data, to optimize cognitive performance restoration.

### CROSS-REFERENCE TO RELATED APPLICATIONS

This application claims the benefit of: U.S. Provisional Application Ser. No. 62/863,662, titled “Assessment of Cognitive Fatigue from a Head Mounted System,” filed by Ben Wisbey and Guillaume Mathias, on Jun. 19, 2019; and, U.S. Provisional Application Ser. No. 62/863,685, titled “Error Risk Detection From Head Mounted Physiological Sensors,” filed by Ben Wisbey and Guillaume Mathias, on Jun. 19, 2019; and, U.S. Provisional Application Ser. No. 62/863,697, titled “Assessment of Cognitive Performance from a Head Mounted System,” filed by Ben Wisbey and Guillaume Mathias, on Jun. 19, 2019.

This application incorporates the entire contents of the above-referenced applications herein by reference.

### TECHNICAL FIELD

Various embodiments relate generally to cognitive performance assessment.

### BACKGROUND

A task is an objective to be completed. In some examples, a task may be work assigned as a part of a person's job or role. In various scenarios, a task may be performed as a recreational activity. For example, a person may enjoy performing well while playing a game, even if the task of playing the game is challenging. Some tasks may be difficult. In an illustrative example, a person engaged in a difficult task may expend substantial mental effort to perform well at the difficult task.

Cognitive load (or mental load) is the mental effort (intensity) used at the current moment to work on the provided task. Cognitive performance is the mental capability available to expend on a task. Cognitive performance and cognitive load may be measured as a function of relationships between brain wave activity determined from an electroencephalogram (EEG), cardiovascular state determined from heart rate variability (HRV) and photoplethysmogram (PPG) data, and machine learning models to predict performance. In some examples, cognitive performance may be measured as a function of cognitive fatigue. Cognitive fatigue (or mental fatigue) is the fatiguing impact of cognitive load applied over time. Cognitive fatigue is a decrease in cognitive resources developing over time on sustained cognitive demands. Achieving a high level of task performance relies on effective levels of cognitive performance, and manageable levels of cognitive fatigue. Cognitive performance may be evaluated as a function of an error in response to a challenge, a response time, quality of output, or volume of output.

Reduced cognitive performance or elevated levels of cognitive fatigue may have a negative performance impact on activities such as computer games, sports, and many occupations including creative and development work. In various scenarios, a person performing a task may be unaware of the risk their level of available cognitive resources may impact cognitive function, including cognitive fatigue, may have on the person's task performance. In an illustrative example, the consequence of poor task performance may be severe. Participants of these activities may have little, or no, insight into their level of cognitive function, load or fatigue.

### SUMMARY

Apparatus and associated methods relate to capturing physiological data from a sensor configured in a user's wearable device while the user performs a task, individualizing the physiological data to the user based on comparison with historical user physiological data, measuring the user's cognitive load determined as a function of the individualized physiological data, and automatically notifying the user of cognitive fatigue detected based on evaluating the measured cognitive load as a function of time. In an illustrative example, the wearable device may be a gaming headset. The measured cognitive load may be, for example, determined as a function of electroencephalograph or heart rate variability data captured from a user while the user performs a task. Some examples may provide recovery recommendations based on the detected cognitive fatigue. Various embodiments may advantageously recommend a recovery schedule determined as a function of a user's historical physiological data, to optimize cognitive performance restoration.

Apparatus and associated methods relate to capturing physiological data from a sensor configured in a user's wearable device while the user performs a task, measuring the user's mental performance determined as a function of the captured physiological data, predicting the user's risk of making an error while performing the task determined as a function of measured mental performance and reference mental performance, and automatically notifying the user of an impending error based on the risk. In an illustrative example, the wearable device may be a gaming headset. The measured mental performance may be, for example, determined as a function of electroencephalograph data captured from a user while the user performs a task. Some examples may interactively provide a live task performance error prediction, based on predicting error risk determined as a function of measured mental performance and a predictive analytic model trained based on reference mental performance associated with a similar task.

Apparatus and associated methods relate to capturing physiological data from a sensor configured in a user's wearable device while the user performs a task, measuring the user's mental performance determined as a function of the captured physiological data, predicting the user's task performance and response time in performing the task determined as a function of measured mental performance and reference mental performance, and providing real time feedback to the user on the expected outcome of their upcoming performance. In an illustrative example, the wearable device may be a gaming headset. The measured mental performance may be, for example, determined as a function of electroencephalograph data captured from a user while the user performs a task. Some examples may interactively provide a live performance score, based on performance prediction determined as a function of measured mental performance and a predictive analytic model trained based on reference mental performance associated with similar tasks.

Apparatus and associated methods relate to storing physiological data captured from a sensor configured in a user's wearable device during a task performance by the user, determining if the task performance is complete, and, in response to determining the task performance is complete: individualizing the physiological data stored during the completed task performance to the user based on comparison with historical user physiological data, measuring the user's cognitive function based on the individualized stored physiological data, and, reporting the user's cognitive fatigue determined based on evaluating the measured cognitive load as a function of time. In an illustrative example, the task performance may be the user playing a game. The user's cognitive function may be measured, for example, to provide the user with retrospective feedback concerning the user's completed task performance. Various embodiments may advantageously provide a gamer between matches with feedback concerning if the gamer should continue to play, based on evaluating data captured during a completed game performance, thereby permitting the gamer to determine if the gamer can continue to play well, based on the data evaluated for the completed game. Some embodiments may present to a gamer a review of the gamer's mental performance evaluated for a completed game. In some implementations, a gamer may be provided with a review of the gamer's mental performance for a completed game, compared to one or more previous games.

In one aspect, the disclosure provides a computer-implemented process to assess task performance, the process comprising: capturing physiological data from a sensor configured in a user's wearable device while the user performs a task; individualizing the physiological data to the user based on comparison with historical user physiological data; measuring the user's cognitive function determined as a function of the individualized physiological data; and, automatically notifying the user of cognitive fatigue detected based on evaluating the measured cognitive load as a function of time.

In one embodiment, the wearable device further comprises a gaming headset.

In another embodiment, the task further comprises playing a game.

In another embodiment, the sensor further comprises an EEG sensor and the physiological data further comprises a signal encoding the user's brain activity.

In another embodiment, the physiological data further comprises HRV data encoding the user's cardiovascular activity.

In another embodiment, measuring the user's cognitive function further comprises evaluating the user's performance based on a mental function metric.

In another embodiment, the mental function metric further comprises power.

In another embodiment, the mental function metric further comprises pressure.

In another embodiment, notifying the user further comprises sending a notification from the user's wearable device to a mobile app configured in another device.

In another aspect, the disclosure provides a computer-implemented process to assess gaming performance, the process comprising: capturing live physiological data from a sensor configured in a user's gaming headset while the user plays a game, wherein the live physiological data comprises EEG and HRV data; individualizing the live physiological data to the user based on comparison with historical user physiological data; measuring the user's cognitive function determined as a function of: the individualized physiological data; a plurality of mental function metrics; and, a predictive analytic model trained with reference physiological data representative of a population of users playing a similar game; and, automatically notifying the user of cognitive fatigue detected based on evaluating the measured cognitive function as a function of time.

In one embodiment, the live physiological data further comprises PPG data.

In another embodiment, the historical user physiological data further comprises data selected from the group consisting of EEG, HRV, and PPG.

In another embodiment, the plurality of mental function metrics further comprise power, and pressure.

In another embodiment, the predictive analytic model further comprises an RDF.

In another embodiment, measuring the user's cognitive function further comprises training an individualized predictive analytic model based on the individualized physiological data and the reference physiological data.

In another embodiment, notifying the user of cognitive fatigue further comprises triggering an indication visible to the user in the user's in-game field of view.

In another aspect, the disclosure provides a computer-implemented process to assess gaming performance, the process comprising: capturing live physiological data from a sensor configured in a user's gaming headset while the user plays a game, wherein the live physiological data comprises EEG, HRV, and PPG data; individualizing the live physiological data to the user based on comparison with historical user physiological data, wherein the historical physiological data comprises EEG, HRV, and PPG data; training an individualized predictive analytic model based on a baseline predictive analytic model, the individualized physiological data, and reference physiological data representative of a population of users playing a similar game; measuring the user's cognitive load determined as a function of: the individualized physiological data; a plurality of mental function metrics; and, the individualized predictive analytic model; and, automatically notifying the user of cognitive fatigue detected based on evaluating the measured cognitive load as a function of time.

In one embodiment, training the individualized predictive analytic model further comprises a controlled training technique.

In another embodiment, capturing live physiological data from the sensor further comprises artifact correction.

In another embodiment, the process further comprises a sensor location in accordance with the International 10-20 system.

In another aspect, the disclosure provides a computer-implemented process to assess mental performance, the process comprising: storing physiological data captured from a sensor configured in a user's wearable device during a task performance by the user; determining if the task performance is complete; in response to determining the task performance is complete: individualizing the physiological data stored during the completed task performance to the user based on comparison with historical user physiological data; measuring the user's cognitive function based on the individualized stored physiological data; and, reporting the user's cognitive fatigue determined based on evaluating the measured cognitive load as a function of time.

In one embodiment, the task performance further comprises the user playing a game.

In another embodiment, determining if the task performance is complete further comprises determining if at least a predetermined portion of the task is complete.

In another embodiment, the predetermined portion of the task may be a portion of a game.

In another embodiment, reporting the user's cognitive fatigue further comprises reporting the cognitive fatigue to the user when the user is between task performances.

In another embodiment, reporting a gamer's cognitive fatigue further comprises providing feedback to the gamer when the gamer is between matches, wherein the feedback concerns if the gamer should continue to play, based on evaluating the gamer's mental performance to determine if the gamer can continue to play well.

In another embodiment, reporting the user's cognitive fatigue further comprises presenting the user with a review of the user's mental performance for the completed task performance.

In another embodiment, reporting the user's cognitive fatigue further comprises presenting the user with a review of the user's mental performance for the completed task performance compared to the user's mental performance for one or more previously completed task performance.

In another embodiment, reporting the user's cognitive fatigue further comprises providing the user feedback concerning the user's mental performance while the user performed the completed task.

In another embodiment, reporting the user's cognitive fatigue further comprises the user's mental performance evaluated as a function of the user's mental performance measured based on user performance of at least one task previous to the completed task.

In another embodiment, reporting a gamer's cognitive fatigue further comprises the gamer's mental performance evaluated as a function of the gamer's mental performance measured based on performance of at least one game previous to the completed game.

In another embodiment, wherein reporting the user's cognitive fatigue further comprises providing the user a prediction of the user's mental performance during a future task performance.

An embodiment apparatus or process may employ sensors (for example, EEG and PPG for HRV) embedded in a gaming headset, or other device, to capture data on the cognitive performance of a gamer wearing the headset or other device paired or connected to the headset. The sensors may be configured as single channel dry EEG on the frontal lobe, and PPG on the temple or forehead. The sensor data may be analyzed in real time to provide feedback to the user. An embodiment apparatus or process may provide detailed feedback to the user via an app configured in a mobile device or another device paired or connected to the user's gaming headset, while summary feedback may be via vibration of a game controller, LED on the headset, or headset audio. In an illustrative example, an embodiment implementation may employ one or more mental function metric to evaluate the sensor data and assess the mental performance of the gamer wearing the headset or other device paired or connected to the headset. The one or more mental function metric employed by the headset or other device paired or connected to the headset may include, for example, Power (fatigue), Pressure (intensity+stress), Focus (concentration), Awareness, and overall Performance (combination of Power, Pressure, Focus, and Awareness). An embodiment design may provide feedback assisting the user to understand if they should compete or practice; for example, if they are likely to perform poorly, the system may provide relevant feedback to encourage them to address the risk (for example, low focus) or take a break (for example, low power).

An embodiment apparatus or process may employ machine learning techniques to improve the accuracy or usefulness of user performance assessment. For example, training the model may include matching captured sensor data to quantifiable testing results including, for example, time on task, work vs rest, error vs success, response time, and the like. The model may be trained based on a controlled training technique, wherein various controlled tests may be conducted, focusing on inducing fatigue with lengthy time on task, having repetitive challenges, and mixing short breaks with challenges. The model may also be trained and/or validated using gaming data matched with sensor data. Such practical training may involve the user playing and tapping an in-app button on positive or negative outcomes. Sensor data may be matched to video recorded game activity, permitting graded practical training incorporating the subjective nature of the user-reported game outcome into game testing and allowing game activity to be rated in more detail. Trained models may be applied across all users as the input data has been personalized prior to training the model. The models may be continually improved as the user uses the system. The system will capture the data, and the user, or the gaming device, will feedback the outcome so the model can continue learning.

An embodiment apparatus or process may capture and analyze EEG data based on wave frequency (delta, theta, alpha, beta, gamma). ECG or PPG data may be captured as inter-beat-interval (RR from ECG) in milliseconds between consecutive beats, then undergoing artifact correction before heart rate variability is assessed. An embodiment system may begin with a generic profile and gradually learn an individual user's personal profile as more data is captured from the individual user. This personalization of the data helps to ensure all data is relevant to the individual user. The data is then processed by a series of equations, ratios and basic analysis. Some metrics are based on output at this stage, including Focus and Awareness. The analyzed data is then run through a machine learning model, and the machine learning model output determined as a function of the analyzed data is used to determine Power and Pressure. The machine learning predictions undergo basic post-processing to apply the data to a given time window, and also normalize the distribution for that individual user. Some metrics are a combination of data. For example, Pressure is a combination of stress from HRV, and intensity from a machine learning model. Performance is also a metric that combines various other metric data. Each metric has an equation based on that metric's impact on ranked game performance. Metric scores are combined for overall performance.

An embodiment apparatus or process may employ algorithms that are hardware agnostic. In an illustrative example, the algorithms may adapt and re-train to various hardware or sensor types or configuration that may be advantageous to a particular embodiment. For example, a single channel EEG may be advantageous for various practical reasons, however the disclosed algorithms could be applied with even more accuracy to 24-channel EEG.

Various embodiment apparatus or process implementations may be configured or deployed in scenarios including, for example, gaming, education, workplace safety, driving, or workplace productivity.

An embodiment apparatus or process may capture data from sensors and live stream this data via Bluetooth to a phone or another device paired or connected to the phone. The phone app will then provide live feedback. The phone will in turn connect to a server to store data, which is where data will be pulled for historical reference and comparison, and ongoing model learning can take place. Some embodiment designs may capture data from sensors and process the captured data to provide retrospective feedback to a user. In an illustrative example, some designs may provide a gamer between matches in a gaming session with feedback concerning if the gamer should continue to play, based on the system determining if the gamer's data show the gamer can continue to play well. In another example, various implementations may provide a gamer that finished a gaming session with a review of the gamer's mental performance, and compare the gamer's mental performance in the finished gaming session to one or more previous gaming session.

Alternative approaches may include data from a WiFi enabled headset communicating directly with the server. Another alternative may be the sensors in the headset communicating directly with the gaming device (for example, Xbox® or PC) which in turn communicates with the server.

In illustrative non-limiting examples, various user mental function metrics determined by an embodiment apparatus or process from sensor data may include, for example, Power, Focus, Awareness, Pressure, Mental intensity, Stress, and Performance.

In an illustrative non-limiting example, Power may be determined using personalized variables to predict mental fatigue using a machine learning model, with post-processing of the predicted value for improved accuracy. For example, Power determination may use 8 feature variables personalized to the users normal range. In an illustrative example, given that fatigue may be a slow changing measure, each feature variable may be assessed over a rolling time window, such as, for example, 5 minutes.

Power is a prediction of mental fatigue based on EEG and HRV. In an illustrative example, Power may be scaled between 0 and 100 in arbitrary units which may be based on the user's normal range. Low Power indicates high fatigue, and provides feedback on the user's capacity to perform. When Power is low, it is less likely that the user will be able to sustain high levels of focus/concentration and thus performance will decrease. In an illustrative example, Power may be predicted using an Extreme Gradient Boosting (XGB) machine learning model with eight feature variables. Power may be predicted using other machine learning model types, as described herein. In an illustrative example describing Power prediction using an XGB with eight feature variables, seven of the feature variables may be sourced from EEG and one feature variable may be HRV. In this example, given the slow changing nature of fatigue, each of the EEG variables may be sampled over a 5 minute period and personalized to the user's normal range using a Z-score. The EEG variables are based on brain wave activity categorized into Delta, Theta, Low Alpha, High Alpha, Low Beta, High Beta, Low Gamma and Mid Gamma frequency ranges. The variables comprise absolute values, ratios, weighted ratios, and mean frequency. HRV uses RMSSD sampled during a 2 minute window prior to being personalized. The predicted value from the machine learning model then undergoes post-processing for improved accuracy, prior to being normalized based on the user's normal fatigue range. The machine learning model is trained using controlled mental tests conducted over fixed periods such as 1 hour. These tests include multiple object tracking, response time test, and a color shape test. Each test captures response accuracy and duration, with time on task providing a proxy for fatigue.

In an illustrative non-limiting example, Focus may be determined using a weighted average of short term beta activity to measure the user's concentration, based on a weighted average of beta wave activity over a period of time, for example, 5 seconds.

Focus may be determined using beta activity derived from EEG to determine the level of concentration the user has dedicated to the activity, which may be reported to the user as a value between 0 and 100 relative to the user's normal range. Focus measures the level of dedicated concentration given to the specific activity. Focus is closely related to performance with optimal performance associated with higher focus levels, and more errors occurring when focus is lower. A Focus value may be calculated from the level of beta activity relative to theta and alpha activity during a predetermined time period or measurement time window, such as, for example, a five second time period or time window. In an illustrative example, a Focus measurement may be weighted to prioritize the most recent data captured in the five second window. For example, distribution may then be spread more evenly using a cubed root method, prior to the value being individualized to the user through use of a z-score. The relationship between Focus and mental performance may be validated using both controlled tests and video games with game play subjectively graded. Controlled tests provide objective measures of performance using response accuracy and response time. Strong statistical relationships are evident between Focus and performance in both controlled tests and video games.

In an illustrative non-limiting example, Awareness may be determined using a weighted average of short term alpha activity over a period of time, to measure the user's mental awareness.

Awareness may be determined using alpha activity derived from EEG to determine the level of mental relaxation the user maintains during an activity. Awareness may be reported to the user as a value between 0 and 100 relative to the user's normal range. Awareness is an assessment of the user's ability to consume and interpret external activity. High levels of Focus often result in narrowed concentration and an inability to recognize broader content, while a relaxed mental state often allows for an increased level of perception. Awareness is an assessment of this level of perception. In an illustrative example, the Awareness value is calculated from the level of alpha activity relative to theta and beta activity during a five second period. In this example, the Awareness measurement is weighted to prioritize the most recent data captured in the five second window. For example, the measurement distribution may then be spread more evenly using a cubed root method, prior to the value being individualized to the user through use of a z-score.

In an illustrative non-limiting example, Pressure may be determined based on combining two components: Stress, based on HRV; and, Mental intensity, predicted from a machine learning model using personalized EEG variables.

Pressure may be determined based on combining two measures (stress and intensity) into a single overall value represented as a normalized score between 0 and 100, relative to the individual user. Pressure provides an assessment of the user's overall mental strain, to provide feedback on how much pressure the user is currently under. While this metric may be of interest to the user, the intensity component is related to an individual's mental performance, with performance increasing when intensity is high. In this example, one component of Pressure is stress, which is based on the user's HRV relative to their normal HRV range. RMSSD during a 2 minute window may be used to assess HRV. In this example, the other component of Pressure is mental intensity which may be predicted using an Extreme Gradient Boosting machine learning model, with 3 EEG feature variables. Intensity may be determined using three EEG variables captured over a 10 second window to train the machine learning model. The rapid changing nature of mental intensity means short windows are needed. In this example, the EEG variables used to determine Intensity are based on brain wave activity categorized into Delta, Theta, Low Alpha, High Alpha, Low Beta, High Beta, Low Gamma and Mid Gamma frequency ranges. In this example, the variables include ratios and weighted ratios. In this example, the predicted value from the machine learning model is smoothed over a 30 second period to reduce volatility and offer more value to the user. In an illustrative example, given the skewed nature of the output values, the data may undergo post-processing to create a more even distribution, prior to being normalized for the individual user. In this example, the machine learning model is trained using controlled mental tests conducted over fixed periods such as 10 minutes and 1 hour. These tests may include multiple object tracking, response time test, and a color shape test. Each test may capture response accuracy and duration, while offering a short break on a fixed schedule. In an illustrative example, such a break versus task comparison provides an opportunity to train the model. In this example, the overall Pressure value is an average of intensity and inverse HRV levels, resulting in Pressure increasing when intensity and stress increase (that is, HRV is reduced). In this example, the Pressure value is then smoothed and normalized to make the measurement more suitable for user interpretation.

In an illustrative non-limiting example, Performance may be evaluated by determining a Performance Score based on a relationship between Power, Focus, Awareness, and mental performance to create a single overall value that represents performance level.

Performance score may provide a single 0-100 value representing the user's overall mental performance level. In an illustrative example, the Performance score may be derived from Power, Focus and Awareness. The Performance score may be based on the relationship of Power, Focus and Awareness with objective performance measures. High Performance scores represent a high likelihood the user will perform well in a game or task. In an illustrative example, Power, Focus and Awareness each have a non-linear relationship with performance, which may be quantified using metric-specific equations to calculate a separate output for each of Power, Focus and Awareness with respect to Performance. The Focus and Awareness values may be averaged, then multiplied by the Power value to develop a single value of performance. This value may then be normalized for the individual user to create the Performance Score. In this example, the relationship between Power, Focus, Awareness, and performance may be determined using a variety of controlled tests and video games to create the equation representing performance for each metric. The controlled tests may use measured response accuracy and response time as performance levels. Additionally, video game performance may be based on captured video of the game with each aspect of the game subjectively rated on a scale, for example, of 1 to 5.

In an illustrative example, Machine Learning may be implemented with each mental function metric personalized to the individual user through statistical methods such as, for example, normalization, and z-scores. In some exemplary scenarios, an approach to machine learning based on mental function metric personalization may be advantageously implemented even when there is limited data available on an individual user. Such a limited-data approach to embodiment machine learning based on personalized mental function metrics may include starting with community based means, standard deviations, minimums, maximums, and percentiles. For example, a 180 minute learning phase for each user may be applied, during which the community values are gradually transitioned to user values. Additionally, widely distributed values may be capped during an initial time period, for example, during the first 30 minutes, to ensure polarized output is avoided when there is limited user data in the individualization process.

In an illustrative example, various details of an embodiment machine learning model may change as the model is trained over time. For example, model parameters that obtain more accurate results when there is limited data available may be slightly different from the model that obtains the best results when more training data is added. In an illustrative example, Power may be based on a prediction from a boosted tree regression model using 8 feature variables selected from over 70 total variables based on variable importance analysis, to optimize accuracy. To maximize model accuracy while also minimizing overfitting, this exemplary Power model may use 50 iterations with a maximum depth of 2. In another illustrative example, Intensity may use a boosted tree regression model with 3 feature variables selected from over 70 total variables based on variable importance analysis, to optimize accuracy. In this exemplary Intensity model, accuracy is maximized, while avoiding overfitting, by using 50 iterations with a maximum depth of 2.

Note that expressions such as ‘feature,’ ‘feature variable,’ and the like, when used in the present disclosure in the context of a mental function metric or sensor data signal description, are intended to be interpreted as referring to one or more predetermined signal characteristic defining the feature or feature variable. For example, a signal feature may define an EEG, HRV, PPG, or other signal characteristic in the time domain or frequency domain, based on amplitude, period, frequency, spectral distribution, correlation or convolution with another signal (for example, a window function as may be known in the art of signal analysis), signal to noise ratio, waveshape, or any other useful signal characteristic known to one of ordinary skill in the arts of signal processing or physiological signal processing.

Various embodiments may achieve one or more advantages. Some examples may increase a user's knowledge of the level of mental energy the user has available to perform at their best. Such increased knowledge of a user's cognitive energy level may be a result of a system configured to measure cognitive fatigue, determining how much mental resource the user has available to continue to achieve a challenging task. Various implementations may increase the accuracy of cognitive fatigue assessment. This facilitation may be a result of EEG and HRV values that are individualized to the user's normal range relative to their baseline values. In an illustrative example, the model may be continually learning as the user captures more data, establishing a more accurate understanding of the user's baseline. Some embodiments may improve a user's ability to avoid deteriorating performance under high fatigue. Such improved avoidance of deteriorating performance may be a result of recovery recommendations triggered in response to predefined thresholds based on the user's individual baseline. In an illustrative example, a gamer about to start a second game may be told they have a high level of cognitive fatigue that will have a negative impact on their performance, and a thirty minute recovery break may be recommended to ensure they can continue to perform at their best when they return. Some embodiments may improve a gamer's ease of access to information about the state of their current mental performance capability. In some embodiments, feedback may be provided directly via a gaming machine or accompanying controller, an accompanying mobile app, or directly via speakers accompanying the sensors (for example, headphones). Some embodiments may improve a gamer's ability to take sufficient time away from gaming to optimize their cognitive performance playing a game. This facilitation may be a result of assessing the gamer's cognitive performance state based on physiological data, such as, for example, electroencephalogram (EEG) or heart rate variability (HRV) data, and providing feedback to the gamer based on the performance state.

In some embodiments, the risk a gamer may perform poorly may be reduced. Such reduced risk of poor game performance may be a result of determining the gamer's current state of cognitive fatigue based on comparing captured electroencephalogram, heart rate variability, or photoplethysmogram (PPG) data with reference data representative of normal levels. Various implementations may help a gamer avoid high levels of cognitive fatigue. This facilitation may be a result of providing the gamer with live feedback determined as a function of the gamer's captured physiological data and a machine learning model trained on reference physiological data. Some embodiments may reduce a gamer's effort optimizing their gaming performance. Such reduced gaming performance optimization effort may be a result of cognitive performance determined as a function of physiological data individualized to a gamer's historical profile data. In an illustrative example, in the case of a new gamer, some designs may implement a learning phase, whereby the system starts with normative values that are replaced with the gamer's personal data as the system is used, permitting the system to deliver on the described experience initially, while becoming increasingly accurate for the individual gamer over time.

Some examples may improve a user's chance of avoiding an error performing a task. Such improved chance of avoiding an error may be a result of a system configured to make the user aware of error risk before an error may occur. Various embodiments may predict when an upcoming error is likely. This facilitation may be a result of a system using binary errors, or poor performances on a scale (for example, percentage), to define an error. In some designs, users may be warned of imminent risk, and take measures to avoid the error. Such warning of imminent risk may be a result of a system modeling poor performance risk on a scale, and applying this model to various distinct but similar tasks, to predict when an error may be likely. Various implementations may provide real time warnings that are highly individualized to a specific user. Such highly individualized warnings may be a result of a system configured to further train a prediction model as user or game feedback is fed back into the model. In an illustrative example, various designs may include a gaming headset that alerts the user when they are at a high risk of error (or performing poorly) so the user can refocus. Some embodiments may improve a gamer's ease of access to information about the state of their current mental performance capability. This facilitation may be a result of alerting the gamer to an increased risk of an error, or providing a mental performance indicator assessing their mental performance. In some embodiments, feedback may be provided directly via a gaming machine or accompanying controller, an accompanying mobile app, or directly via speakers accompanying the sensors (for example, headphones). Such automatic feedback may reduce a user's risk of committing an error playing a game. Some embodiments may improve a gamer's ability to take sufficient time away from gaming to optimize their cognitive performance playing a game. This facilitation may be a result of assessing the gamer's cognitive performance state based on physiological data, such as, for example, electroencephalogram (EEG) or heart rate variability (HRV) data, and providing feedback to the gamer based on the performance state.

In some embodiments, the risk a gamer may make an error in a game may be reduced. Such reduced risk of poor game performance may be a result of determining the gamer's short term risk of an error, or mistake based on comparing captured electroencephalogram, heart rate variability, or photoplethysmogram (PPG) data with reference data representative of normal levels, over a short period of time. Various implementations may help a gamer avoid making an error in playing a game. This facilitation may be a result of providing the gamer with live feedback determined as a function of the gamer's captured physiological data and a machine learning model trained on reference physiological data. Some embodiments may reduce a gamer's effort optimizing their gaming performance. Such reduced gaming performance optimization effort may be a result of cognitive performance determined as a function of physiological data individualized to a gamer's historical profile data. In an illustrative example, in the case of a new gamer, some designs may implement a learning phase, whereby the system starts with normative values that are replaced with the gamer's personal data as the system is used, permitting the system to deliver on the described experience initially, while becoming increasingly accurate for the individual gamer over time. In some embodiments, the risk a gamer may perform poorly in a game may be reduced. Such reduced risk of poor game performance may be a result of determining the gamer's risk of an error, or mistake over a time period required for a game, based on comparing captured electroencephalogram, heart rate variability, or photoplethysmogram (PPG) data with reference data representative of normal levels.

Some examples may improve a user's insight into how ready they may be to perform. Such improved insight into performance readiness may be a result of more accurately predicting upcoming response times for the same task, based on using response time as a measure of performance. Various embodiments may advantageously provide a clear score and recommendation even when task-specific training is not possible or practical. This facilitation may be a result of a predicted performance score generated as a function of a response time prediction, and applying the performance score to subjective activities, or activities that may be similar, but not identical to an original training task. In an illustrative example, a gamer may put on their headset to play a game, and be informed with a performance score predicting how ready they are to perform, permitting them to select whether to compete, practice, or rest, based on the predicted performance score. Some embodiments may improve a gamer's ease of access to information about the state of their current mental performance capability. This facilitation may be a result of alerting the gamer to an increased risk of an error, or providing a mental performance indicator assessing their mental performance. In an illustrative example, the performance indicator assessing the user's mental performance may be based on a measured user response time. In some embodiments, feedback may be provided directly via a gaming machine or accompanying controller, an accompanying mobile app, or directly via speakers accompanying the sensors (for example, headphones). Such automatic feedback may reduce a user's risk of committing an error playing a game. Some embodiments may improve a gamer's ability to take sufficient time away from gaming to optimize their cognitive performance playing a game. This facilitation may be a result of assessing the gamer's cognitive performance state based on physiological data, such as, for example, electroencephalogram (EEG) or heart rate variability (HRV) data, and providing feedback to the gamer based on the performance state.

The details of various embodiments are set forth in the accompanying drawings and the description below. Other features and advantages will be apparent from the description and drawings, and from the claims.

### BRIEF DESCRIPTION OF THE DRAWINGS

FIG. 1 depicts an exemplary head-mounted system in an illustrative operational scenario assessing a user's performance according to a user mental function metric based on capturing physiological data from a sensor configured in a user's wearable device while the user performs a task, in accordance with an embodiment of the present disclosure.

FIG. 2 depicts a schematic view of an exemplary network configured to assess a user's performance according to a user mental function metric based on capturing physiological data from a sensor configured in a user's wearable device while the user performs a task, in accordance with an embodiment of the present disclosure.

FIG. 3 depicts a structural view of an exemplary head-mounted system configured to assess a user's performance according to a user mental function metric based on capturing physiological data from a sensor configured in a user's wearable device while the user performs a task, in accordance with an embodiment of the present disclosure.

FIG. 4 depicts an exemplary process flow of an embodiment UPOE (User Performance Optimization Engine) assessing a user's performance according to a user mental function metric based on capturing physiological data from a sensor configured in a user's wearable device while the user performs a task, in accordance with an aspect of the present disclosure.

FIG. 5 depicts an exemplary process flow of an embodiment UPOE (User Performance Optimization Engine) assessing a user's performance according to a user mental function metric based on capturing physiological data from a sensor configured in a user's wearable device while the user performs a task, in accordance with another aspect of the present disclosure.

FIG. 6 depicts exemplary process steps to assess user performance according to a user mental function metric.

FIGS. 7A-7B together depict exemplary training and usage of an embodiment machine learning model configured to assess user performance according to a user mental function metric.

FIG. 8 depicts an exemplary information flow to assess user performance according to a user mental function metric.

Like reference symbols in the various drawings indicate like elements.

### DETAILED DESCRIPTION OF ILLUSTRATIVE EMBODIMENTS

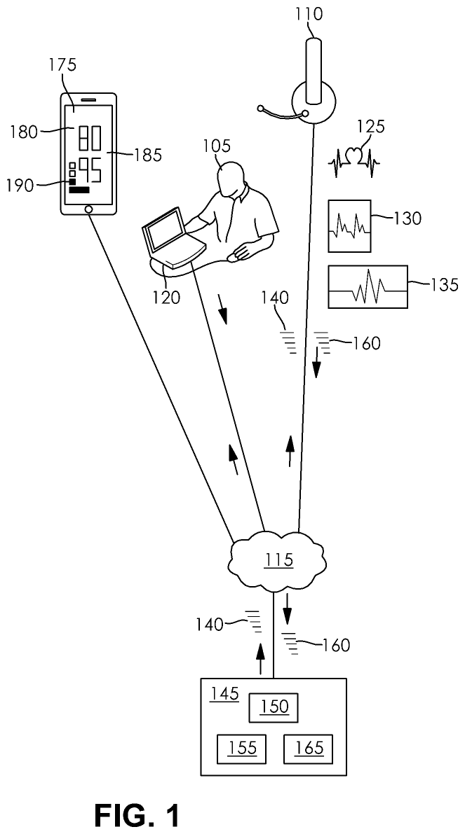

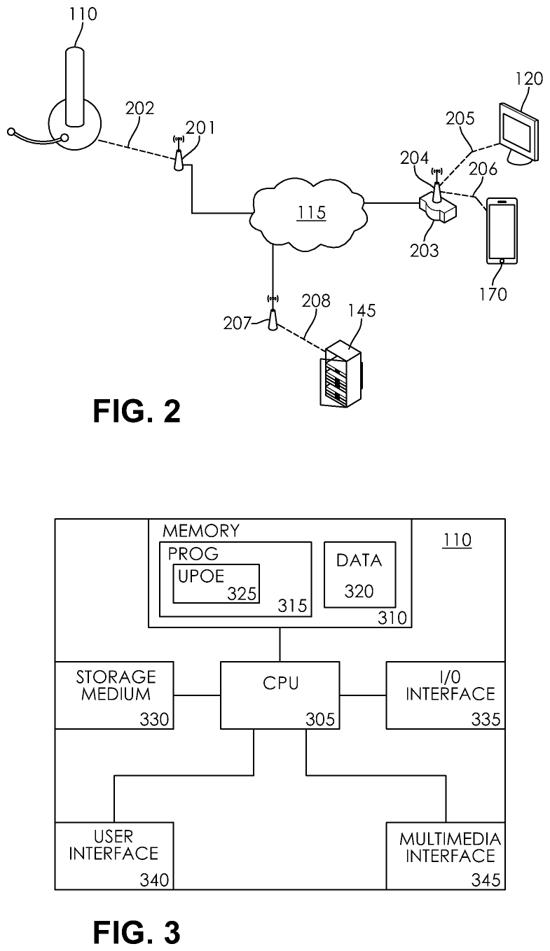

To aid understanding, this document is organized as follows. First, live cognitive performance assessment based on physiological sensor data is briefly introduced with reference to FIG. 1. Then, with reference to FIGS. 2-8, the discussion turns to exemplary embodiments illustrating performance assessment based on evaluating captured physiological sensor data using a mental function metric. Specifically, exemplary performance assessment network, physiological sensor device, cognitive fatigue assessment process, cognitive error risk prediction process, and data flow implementations are disclosed, to explain improvements in cognitive performance, cognitive fatigue, and cognitive error risk assessment technologies.

FIG. 1 depicts an exemplary head-mounted system in an illustrative operational scenario assessing a user's performance according to a user mental function metric based on capturing physiological data from a sensor configured in a user's wearable device while the user performs a task, in accordance with an embodiment of the present disclosure.

In one aspect, the head-mounted system depicted by FIG. 1 may assess a user's cognitive fatigue based on capturing physiological data from a sensor configured in the device while the user performs a task, individualizing the physiological data to the user based on comparison with historical user physiological data, measure the user's cognitive load determined as a function of the individualized physiological data, and automatically notify the user of cognitive fatigue detected based on evaluating the measured cognitive load as a function of time.

In another aspect, the head-mounted system depicted by FIG. 1 may assess a user's error risk based on capturing physiological data from a sensor configured in the device while the user performs a task, measure the user's mental performance determined as a function of the captured physiological data, predict the user's risk of poorly performing the task determined as a function of measured mental performance and reference mental performance, and automatically notify the user of an impending error based on the risk of poor performance.

In another aspect, the head-mounted system depicted by FIG. 1 may assess a user's cognitive performance based on capturing physiological data from a sensor configured in a user's wearable device while the user performs a task, measure the user's mental performance determined as a function of the captured physiological data, predict the user's task performance and response time in performing the task determined as a function of measured mental performance and reference mental performance, and provide real time feedback to the user on the expected outcome of their upcoming performance.

In FIG. 1, the user 105 is a gamer playing a game while using a wearable device configured with a physiological sensor. Some examples may provide real time feedback to the user on how to improve their performance based on a user 105 mental function metric determined based on physiological data captured from the sensor. In one aspect, the user 105 mental function metric may be cognitive performance. In another aspect, the user 105 mental function metric may be error risk. In another aspect, the user 105 mental function metric may be cognitive fatigue. In the depicted embodiment, the wearable device worn by the gamer 105 is the gaming headset 110 operably and communicatively coupled through the network cloud 115 with the gaming system 120. In the depicted example, the gaming headset 110 is configured with one or more physiological sensor adapted to measure a physiological parameter of the gamer 105 physiological response to playing the game. In the illustrated embodiment, the physiological sensor is configured to emit data representative of a gamer 105 physiological response parameter measured by the sensor while the gamer 105 plays the game. In various embodiments, the physiological sensor may include a heart rate variability (HRV) sensor configured in the gaming headset 110. In some embodiments, the physiological sensor may include an electroencephalogram (EEG) sensor configured in the gaming headset 110. In some examples, the physiological sensor may include a photoplethysmogram (PPG) sensor configured in the gaming headset 110. In some implementations, HRV may be determined as a function of heart rate data captured from the PPG sensor. In various embodiments, the gaming headset 110 may include more than one sensor. In the depicted example, the gaming headset 110 determines the gamer 105 EEG 130 based on measurement by the EEG sensor, while the gamer 105 plays the game. In the illustrated example, the gaming headset 110 determines the gamer 105 PPG 135 based on measurement by the PPG sensor, while the gamer 105 plays the game. In the illustrated example, the gaming headset 110 determines the gamer 105 HRV 125 based on the gamer 105 PPG 135. In the illustrated embodiment, the gaming headset 110 retrieves the baseline machine learning model 140 from the cloud server 145 baseline machine learning model database 150. In the depicted embodiment, the cloud server 145 is operably and communicatively coupled with the network cloud 115. In the illustrated embodiment, the baseline machine learning model 140 is an Extreme Gradient Boosting (XGB) model. In some embodiments, the baseline machine learning model 140 may be a neural network. In various designs, the baseline machine learning model may be, for example, based on a Random Decision Forest (RDF), or other machine learning method. In the depicted embodiment, the baseline machine learning model 140 has been trained as a function of reference cognitive performance measurements determined as a function of physiological data associated with the task performed by the user. In some examples, the reference cognitive performance data used to train the baseline machine learning model 140 may be representative of the cognitive performance of a particular population performing the task the user will perform while physiological data is captured from the user. In the illustrated embodiment, the gaming headset 110 sends the gamer 105 physiological data including the HRV 125 data, EEG 130 data, and PPG 135 data to the cloud server 145. In the depicted embodiment, the cloud server 145 stores the gamer 105 physiological data including the HRV 125, EEG 130, and PPG 135 to the user profile database 155. In various examples, the baseline machine learning model 140 may be trained as a function of the historical physiological data stored in the user profile database 155. In some embodiments, the cloud server 145 may be omitted, and the machine learning models may be embedded in the headset, or the mobile app. In the depicted embodiment, the gaming headset 110 measures the gamer 105 cognitive performance determined as a function of the physiological data captured by the gaming headset 110 while the gamer 105 plays the game. In the illustrated embodiment, the gaming headset 110 predicts the gamer 105 cognitive fatigue risk determined as a function of the baseline machine learning model 140, reference cognitive performance, and the measured cognitive performance. In another embodiment, the gaming headset 110 may predict the gamer 105 poor performance risk determined as a function of the baseline machine learning model 140, reference cognitive performance, and the measured cognitive performance. In the depicted embodiment, the gaming headset 110 creates the updated machine learning model 160 based on training the baseline machine learning model 140 as a function of the predicted cognitive performance and the HRV 125, EEG 130, and PPG 135 data. In the illustrated embodiment, the gaming headset 110 sends the updated machine learning model 160 to the cloud server 145 to be stored on the enhanced machine learning model database 165. In the illustrated embodiment, the user 105 employs the mobile device 170 to monitor cognitive performance while playing the game. In the illustrated example, the mobile device 170 is communicatively and operably coupled with the network cloud 115. In various examples, the mobile device 170 may be offline, without a connection to the network cloud 115. In some examples, the mobile device 170 may be operably and communicatively coupled with the gaming headset 110 by a communication link. In the depicted example, the gaming headset 110 automatically sends alerts to the mobile device 170 to notify the user 105 of an impending error, predicted performance level and state of cognitive fatigue based on the cognitive function measurements. In some examples, the gaming headset 110 may automatically send alerts to the mobile device 170 to notify the user 105 of an impending poor performance, predicted performance level and state of cognitive fatigue based on the cognitive function measurements. In the depicted embodiment, the mobile device 170 includes the mobile app 175. In the illustrated embodiment, the mobile app 175 is configured to present the user with cognitive performance alerts and status received from the gaming headset 110. In the depicted embodiment, the mobile app 175 displays the user 105 cognitive load 180 received from the gaming headset 110. In the illustrated embodiment, the mobile app 175 also displays the user 105 cognitive performance 185 received from the gaming headset 110. In the depicted embodiment, the mobile app 175 displays the user 105 cognitive fatigue level, error risk and overall performance level 190 received from the gaming headset 110. In various examples, the user 105 may optimize their task performance based on live feedback received from the gaming headset 110 while playing the game. In some examples, communication with the cloud server 145 may be optional. In an illustrative example, various embodiment cognitive performance measurements may be performed directly with the mobile app 175, the gaming system 120, or onboard the gaming headset 110, and any of these devices may then optionally communicate with the cloud server 145 if present.

FIG. 2 depicts a schematic view of an exemplary network configured to assess a user's performance according to a user mental function metric based on capturing physiological data from a sensor configured in a user's wearable device while the user performs a task, in accordance with an embodiment of the present disclosure.

In one aspect, the cognitive performance assessment network depicted by FIG. 2 may be configured to assess a user's cognitive fatigue based on capturing physiological data from a sensor configured in a user's wearable device while the user performs a task, individualize the physiological data to the user based on comparison with historical user physiological data, measure the user's cognitive load determined as a function of the individualized physiological data, and automatically notify the user of cognitive fatigue detected based on evaluating the measured cognitive load as a function of time.

In another aspect, the cognitive performance assessment network depicted by FIG. 2 may be configured to assess a user's error risk based on capturing physiological data from a sensor configured in the device while the user performs a task, measure the user's mental performance determined as a function of the captured physiological data, predict the user's risk of poorly performing the task determined as a function of measured mental performance and reference mental performance, and automatically notify the user of an impending error based on the risk of poor performance.

In another aspect, the cognitive performance assessment network depicted by FIG. 2 may be configured to assess a user's cognitive performance based on capturing physiological data from a sensor configured in a user's wearable device while the user performs a task, measure the user's mental performance determined as a function of the captured physiological data, predict the user's task performance and response time in performing the task determined as a function of measured mental performance and reference mental performance, and provide real time feedback to the user on the expected outcome of their upcoming performance.

In FIG. 2, according to an exemplary embodiment of the present disclosure, data may be transferred to the system, stored by the system and/or transferred by the system to users of the system across local area networks (LANs) or wide area networks (WANs). In accordance with various embodiments, the system may include numerous servers, data mining hardware, computing devices, or any combination thereof, communicatively connected across one or more LANs and/or WANs. One of ordinary skill in the art would appreciate that there are numerous manners in which the system could be configured, and embodiments of the present disclosure are contemplated for use with any configuration. Referring to FIG. 2, a schematic overview of a system in accordance with an embodiment of the present disclosure is shown. In the depicted embodiment, an exemplary system includes the exemplary gaming headset 110 configured to determine a user mental function metric measured as a function of physiological data captured from sensors in the gaming headset 110. In one aspect, the user mental function metric may be cognitive performance. In another aspect, the user mental function metric may be error risk. In another aspect, the user mental function metric may be cognitive fatigue. In the illustrated embodiment, the cloud server 145 is a computing device configured to provide storage for and access to machine learning models and physiological data. In the depicted embodiment, the mobile device 170 is a computing device configured to monitor the gamer 105 cognitive performance, error risk, or cognitive fatigue, based on alerts received from the gaming headset 110. In the illustrated embodiment, the gaming system 120 is a computing device configured to host games played by the gamer 105. In the illustrated embodiment, the gaming headset 110 is communicatively and operably coupled by the wireless access point 201 and the wireless link 202 with the network cloud 115 (for example, the Internet) to send, retrieve, or manipulate information in storage devices, servers, and network components, and exchange information with various other systems and devices via the network cloud 115.

In another embodiment, the gaming headset 110 may be paired or connected to the mobile device 170, to communicate directly with the mobile device 170. For example, the network connection between the gaming headset 110 and the network cloud 115 may be omitted, and the gaming headset 110 may communicate directly with the mobile device 170, permitting the gaming headset 110 to connect through the mobile device 170 to the network cloud 115.

In the depicted example, the illustrative system includes the router 203 communicatively and operably coupled with the wireless access point 204 to communicatively and operably couple the gaming system 120 to the network cloud 115 via the communication link 205. In the illustrated example, the router 203 and the wireless access point 204 also communicatively and operably couple the mobile device 170 to the network cloud 115 via the communication link 206. In the depicted embodiment, the cloud server 145 is communicatively and operably coupled with the network cloud 115 by the wireless access point 207 and the wireless communication link 208. In various examples, one or more of: the gaming headset 110, cloud server 145, mobile device 170, or gaming system 120 may include an application server configured to store or provide access to information used by the system. In various embodiments, one or more application server may retrieve or manipulate information in storage devices and exchange information through the network cloud 115. In some examples, one or more of: the gaming headset 110, cloud server 145, mobile device 170, or gaming system 120 may include various applications implemented as processor-executable program instructions. In some embodiments, various processor-executable program instruction applications may also be used to manipulate information stored remotely and process and analyze data stored remotely across the network cloud 115 (for example, the Internet). According to an exemplary embodiment, as shown in FIG. 2, exchange of information through the network cloud 115 or other network may occur through one or more high speed connections. In some cases, high speed connections may be over-the-air (OTA), passed through networked systems, directly connected to one or more network cloud 115 or directed through one or more router. In various implementations, one or more router may be optional, and other embodiments in accordance with the present disclosure may or may not utilize one or more router. One of ordinary skill in the art would appreciate that there are numerous ways any or all of the depicted devices may connect with the network cloud 115 for the exchange of information, and embodiments of the present disclosure are contemplated for use with any method for connecting to networks for the purpose of exchanging information. Further, while this application may refer to high speed connections, embodiments of the present disclosure may be utilized with connections of any useful speed. In an illustrative example, components or modules of the system may connect to one or more of: the gaming headset 110, cloud server 145, mobile device 170, or gaming system 120 via the network cloud 115 or other network in numerous ways. For instance, a component or module may connect to the system i) through a computing device directly connected to the network cloud 115, ii) through a computing device connected to the network cloud 115 through a routing device, or iii) through a computing device connected to a wireless access point. One of ordinary skill in the art will appreciate that there are numerous ways that a component or module may connect to a device via network cloud 115 or other network, and embodiments of the present disclosure are contemplated for use with any network connection method. In various examples, one or more of: the gaming headset 110, cloud server 145, mobile device 170, or gaming system 120 could include a personal computing device, such as a smartphone, tablet computer, wearable computing device, cloud-based computing device, virtual computing device, or desktop computing device, configured to operate as a host for other computing devices to connect to. In some examples, one or more communications means of the system may be any circuitry or other means for communicating data over one or more networks or to one or more peripheral devices attached to the system, or to a system module or component. Appropriate communications means may include, but are not limited to, wireless connections, wired connections, cellular connections, data port connections, Bluetooth® connections, near field communications (NFC) connections, or any combination thereof. One of ordinary skill in the art will appreciate that there are numerous communications means that may be utilized with embodiments of the present disclosure, and embodiments of the present disclosure are contemplated for use with any communications means.

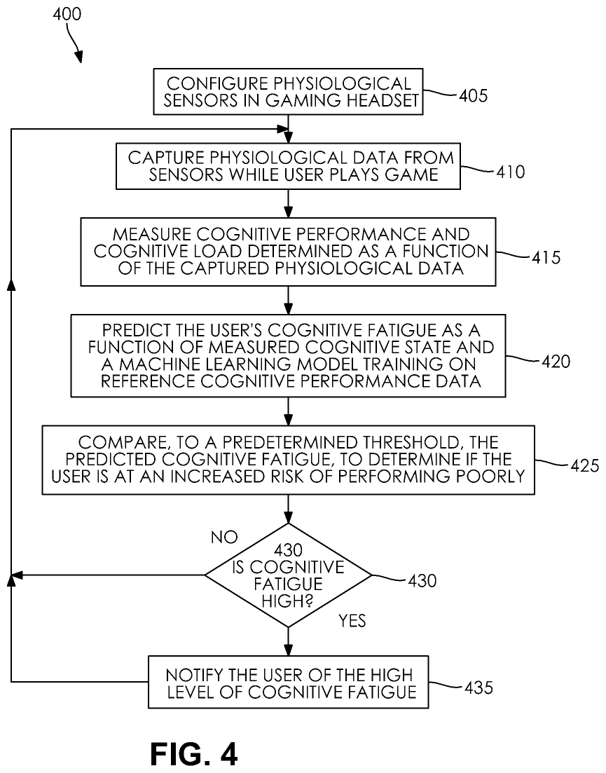

FIG. 3 depicts a structural view of an exemplary head-mounted system configured to assess a user's performance according to a user mental function metric based on capturing physiological data from a sensor configured in a user's wearable device while the user performs a task, in accordance with an embodiment of the present disclosure.

In FIG. 3, the block diagram of the exemplary gaming headset 110 includes processor 305 and memory 310. The processor 305 is in electrical communication with the memory 310. The depicted memory 310 includes program memory 315 and data memory 320. The depicted program memory 315 includes processor-executable program instructions implementing the UPOE (User Performance Optimization Engine) 325.

In one aspect, the user mental function metric may be cognitive fatigue, and the UPOE 325 may be configured to assess the user's cognitive fatigue based on capturing physiological data from a sensor configured in the user's wearable device while the user performs a task, individualize the physiological data to the user based on comparison with historical user physiological data, measure the user's cognitive load determined as a function of the individualized physiological data, and automatically notify the user of cognitive fatigue detected based on evaluating the measured cognitive load as a function of time.

In another aspect, the user mental function metric may be error risk, and the UPOE 325 may be configured to assess the user's error risk based on capturing physiological data from a sensor configured in the device while the user performs a task, measure the user's mental performance determined as a function of the captured physiological data, predict the user's risk of poorly performing the task determined as a function of measured mental performance and reference mental performance, and automatically notify the user of an impending error based on the risk of poor performance.

In another aspect, the user mental function metric may be cognitive performance, and the UPOE 325 may be configured to assess a user's cognitive performance based on capturing physiological data from a sensor configured in a user's wearable device while the user performs a task, measure the user's mental performance determined as a function of the captured physiological data, predict the user's task performance and response time in performing the task determined as a function of measured mental performance and reference mental performance, and provide real time feedback to the user on the expected outcome of their upcoming performance.

In some embodiments, the illustrated program memory 315 may include processor-executable program instructions configured to implement an OS (Operating System). In various embodiments, the OS may include processor executable program instructions configured to implement various operations when executed by the processor 305. In some embodiments, the OS may be omitted. In some embodiments, the illustrated program memory 315 may include processor-executable program instructions configured to implement various Application Software. In various embodiments, the Application Software may include processor executable program instructions configured to implement various operations when executed by the processor 305. In some embodiments, the Application Software may be omitted. In the depicted embodiment, the processor 305 is communicatively and operably coupled with the storage medium 330. In the depicted embodiment, the processor 305 is communicatively and operably coupled with the I/O (Input/Output) interface 335. In the depicted embodiment, the I/O interface 335 includes a network interface. In various implementations, the network interface may be a wireless network interface. In some designs, the network interface may be a Wi-Fi interface. In some embodiments, the network interface may be a Bluetooth interface. In an illustrative example, the gaming headset 110 may include more than one network interface. In some designs, the network interface may be a wireline interface. In some designs, the network interface may be omitted. In the depicted embodiment, the processor 305 is communicatively and operably coupled with the user interface 340. In various implementations, the user interface 340 may be adapted to receive input from a user or send output to a user. In some embodiments, the user interface 340 may be adapted to an input-only or output-only user interface mode. In various implementations, the user interface 340 may include an imaging display. In some embodiments, the user interface 340 may include an audio interface. In some designs, the audio interface may include an audio input. In various designs, the audio interface may include an audio output. In some implementations, the user interface 340 may be touch-sensitive. In some designs, the gaming headset 110 may include an accelerometer operably coupled with the processor 305. In various embodiments, the gaming headset 110 may include a GPS module operably coupled with the processor 305. In some implementations, the gaming headset 110 may include an EEG sensor module operably coupled with the processor 305. In some embodiments, the gaming headset 110 may include an HRV sensor module operably coupled with the processor 305. In some designs, the gaming headset 110 may include a PPG sensor module operably coupled with the processor 305. Various embodiment gaming headset 110 designs may include a gyroscope module operably coupled with the processor 305. In some implementations, the gaming headset 110 may include a motion sensor module operably coupled with the processor 305. In an illustrative example, the gaming headset 110 may include a magnetometer operably coupled with the processor 305. In some embodiments the user interface 340 may include an input sensor array. In various implementations, the input sensor array may include one or more imaging sensor. In various designs, the input sensor array may include one or more audio transducer. In some implementations, the input sensor array may include a radio-frequency detector. In an illustrative example, the input sensor array may include an ultrasonic audio transducer. In some embodiments, the input sensor array may include image sensing subsystems or modules configurable by the processor 305 to be adapted to provide image input capability, image output capability, image sampling, spectral image analysis, correlation, autocorrelation, Fourier transforms, image buffering, image filtering operations including adjusting frequency response and attenuation characteristics of spatial domain and frequency domain filters, image recognition, pattern recognition, or anomaly detection. In various implementations, the depicted memory 310 may contain processor executable program instruction modules configurable by the processor 305 to be adapted to provide image input capability, image output capability, image sampling, spectral image analysis, correlation, autocorrelation, Fourier transforms, image buffering, image filtering operations including adjusting frequency response and attenuation characteristics of spatial domain and frequency domain filters, image recognition, pattern recognition, or anomaly detection. In some embodiments, the input sensor array may include audio sensing subsystems or modules configurable by the processor 305 to be adapted to provide audio input capability, audio output capability, audio sampling, spectral audio analysis, correlation, autocorrelation, Fourier transforms, audio buffering, audio filtering operations including adjusting frequency response and attenuation characteristics of temporal domain and frequency domain filters, audio pattern recognition, or anomaly detection. In various implementations, the depicted memory 310 may contain processor executable program instruction modules configurable by the processor 305 to be adapted to provide audio input capability, audio output capability, audio sampling, spectral audio analysis, correlation, autocorrelation, Fourier transforms, audio buffering, audio filtering operations including adjusting frequency response and attenuation characteristics of temporal domain and frequency domain filters, audio pattern recognition, or anomaly detection. In the depicted embodiment, the processor 305 is communicatively and operably coupled with the multimedia interface 345. In the illustrated embodiment, the multimedia interface 345 includes interfaces adapted to input and output of audio, video, and image data. In some embodiments, the multimedia interface 345 may include one or more still image camera or video camera. In various designs, the multimedia interface 345 may include one or more microphone. In some implementations, the multimedia interface 345 may include a wireless communication means configured to operably and communicatively couple the multimedia interface 345 with a multimedia data source or sink external to the gaming headset 110. In various designs, the multimedia interface 345 may include interfaces adapted to send, receive, or process encoded audio or video. In various embodiments, the multimedia interface 345 may include one or more video, image, or audio encoder. In various designs, the multimedia interface 345 may include one or more video, image, or audio decoder. In various implementations, the multimedia interface 345 may include interfaces adapted to send, receive, or process one or more multimedia stream. In various implementations, the multimedia interface 345 may include a GPU. In some embodiments, the multimedia interface 345 may be omitted. Useful examples of the illustrated gaming headset 110 include, but are not limited to, personal computers, servers, tablet PCs, smartphones, or other computing devices. In some embodiments, multiple gaming headset 110 devices may be operably linked to form a computer network in a manner as to distribute and share one or more resources, such as clustered computing devices and server banks/farms. Various examples of such general-purpose multi-unit computer networks suitable for embodiments of the disclosure, their typical configuration and many standardized communication links are well known to one skilled in the art, as explained in more detail in the foregoing FIG. 2 description. In some embodiments, an exemplary gaming headset 110 design may be realized in a distributed implementation. In an illustrative example, some gaming headset 110 designs may be partitioned between a client device, such as, for example, a phone, and, a more powerful server system, as depicted, for example, in FIG. 2. In various designs, a gaming headset 110 partition hosted on a PC or mobile device may choose to delegate some parts of computation, such as, for example, machine learning or deep learning, to a host server. In some embodiments, a client device partition may delegate computation-intensive tasks to a host server to take advantage of a more powerful processor, or to offload excess work. In an illustrative example, some devices may be configured with a mobile chip including an engine adapted to implement specialized processing, such as, for example, neural networks, machine learning, artificial intelligence, image recognition, audio processing, or digital signal processing. In some embodiments, such an engine adapted to specialized processing may have sufficient processing power to implement some features. However, in some embodiments, an exemplary gaming headset 110 may be configured to operate on a device with less processing power, such as, for example, various gaming consoles, which may not have sufficient processor power, or a suitable CPU architecture, to adequately support gaming headset 110. Various embodiment designs configured to operate on a such a device with reduced processor power may work in conjunction with a more powerful server system.

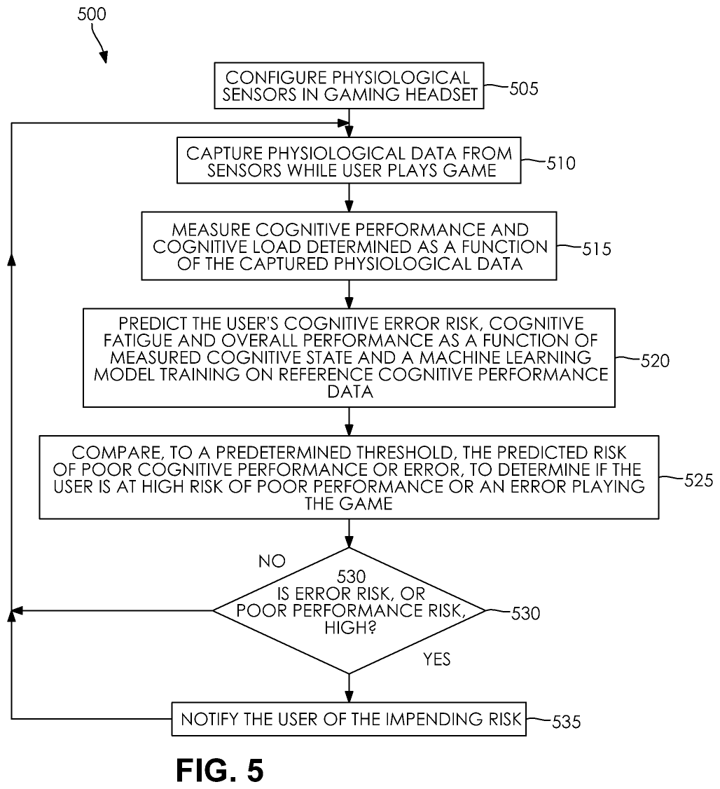

FIG. 4 depicts an exemplary process flow of an embodiment UPOE (User Performance Optimization Engine) assessing a user's performance according to a user mental function metric based on capturing physiological data from a sensor configured in a user's wearable device while the user performs a task, in accordance with an aspect of the present disclosure.

In one aspect, the user mental function metric may be cognitive fatigue, and the UPOE 325 may be configured to assess the user's cognitive fatigue based on capturing physiological data from a sensor configured in the user's wearable device while the user performs a task, individualize the physiological data to the user based on comparison with historical user physiological data, measure the user's cognitive load determined as a function of the individualized physiological data, and automatically notify the user of cognitive fatigue detected based on evaluating the measured cognitive load as a function of time.

The method depicted in FIG. 4 is given from the perspective of the UPOE (User Performance Optimization Engine) 325 implemented via processor-executable program instructions executing on the gaming headset 110 processor 305, depicted in FIG. 3. In the illustrated embodiment, the UPOE 325 executes as program instructions on the processor 305 configured in the UPOE 325 host gaming headset 110, depicted in at least FIG. 1, FIG. 2, and FIG. 3. In some embodiments, the UPOE 325 may execute as a cloud service communicatively and operatively coupled with system services, hardware resources, or software elements local to and/or external to the UPOE 325 host gaming headset 110. The depicted method 400 begins at step 405 with the processor 305 configuring physiological sensors in gaming headset 110 to capture physiological data from a user while the user plays a game. In various designs, the sensors may include EEG, HRV, or PPG physiological sensors and motion sensors, permitting the processor 305 to measure cognitive load, cognitive performance, and cognitive fatigue determined as a function of the sensor data. Then, the method continues at step 410 with the processor 305 capturing physiological data from the sensors while the user plays the game. In some implementations the processor 305 may determine cognitive fatigue based on evaluating the measured cognitive load as a function of time. In various embodiments, the processor 305 may predict the user's risk of cognitive fatigue determined as a function of measured cognitive performance and a predictive analytic model trained based on reference cognitive performance data. The method continues at step 415 with the processor 305 measuring the user's cognitive performance and cognitive load determined as a function of the captured physiological data. The method continues at step 420 with the processor 305 predicting the user's cognitive fatigue as a function of measured cognitive state and a machine learning model training on reference cognitive performance data. The method continues at step 425 with the processor 305 comparing, to a predetermined threshold, the predicted cognitive fatigue, to determine if the user is at an increased risk of performing poorly, based on the comparison. The method continues at step 430 with the processor 305 performing a test to determine if the user's cognitive fatigue is high, based on the comparison performed by the processor 305 at step 425. Upon a determination by the processor 305 at step 430 the user's cognitive fatigue is high, the method continues at step 435 with the processor 305 notifying the user of the high level of cognitive fatigue, and the method continues at step 410 with the processor 305 capturing physiological data from the sensors while the user plays the game. In various embodiments, the processor 305 may notify the user of the high level of cognitive fatigue in various ways. For example, the processor 305 may notify the user of the high level of cognitive fatigue by triggering an audibly or visibly detectable alert on the user's mobile device, gaming headset, or in the game the user is playing. Upon a determination by the processor 305 at step 430 the user's level of cognitive fatigue is not high, the method continues at step 410 with the processor 305 capturing physiological data from the sensors while the user plays the game. In various embodiments, the method may repeat.

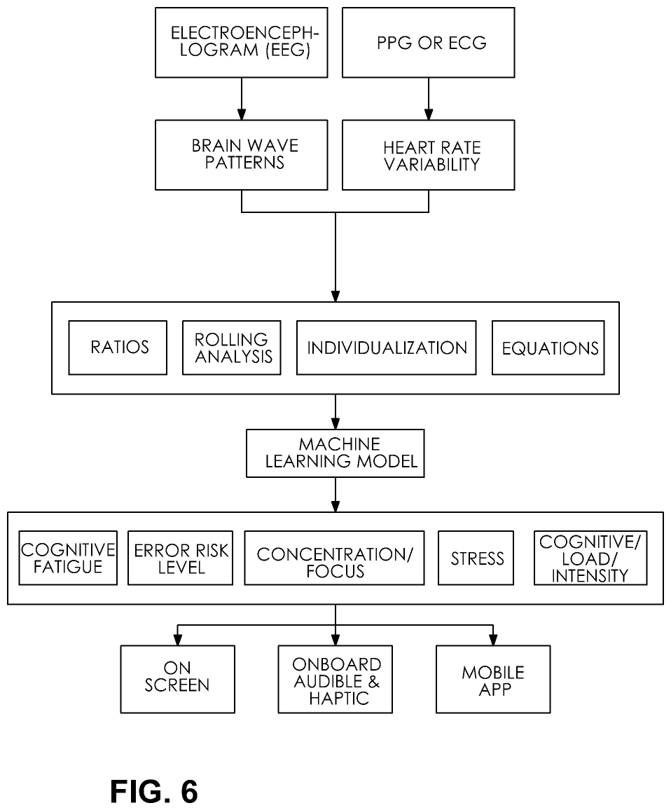

FIG. 5 depicts an exemplary process flow of an embodiment UPOE (User Performance Optimization Engine) assessing a user's performance according to a user mental function metric based on capturing physiological data from a sensor configured in a user's wearable device while the user performs a task, in accordance with another aspect of the present disclosure.

In one aspect, the user mental function metric may be error risk, and the UPOE 325 may be configured to assess the user's error risk based on capturing physiological data from a sensor configured in the device while the user performs a task, measure the user's mental performance determined as a function of the captured physiological data, predict the user's risk of poorly performing the task determined as a function of measured mental performance and reference mental performance, and automatically notify the user of an impending error based on the risk of poor performance.

In another aspect, the user mental function metric may be cognitive performance, and the UPOE 325 may be configured to assess a user's cognitive performance based on capturing physiological data from a sensor configured in a user's wearable device while the user performs a task, measure the user's mental performance determined as a function of the captured physiological data, predict the user's task performance and response time in performing the task determined as a function of measured mental performance and reference mental performance, and provide real time feedback to the user on the expected outcome of their upcoming performance.

The method depicted in FIG. 5 is given from the perspective of the UPOE (User Performance Optimization Engine) 325 implemented via processor-executable program instructions executing on the gaming headset 110 processor 305, depicted in FIG. 3. In the illustrated embodiment, the UPOE 325 executes as program instructions on the processor 305 configured in the UPOE 325 host gaming headset 110, depicted in at least FIG. 1, FIG. 2, and FIG. 3. In some embodiments, the UPOE 325 may execute as a cloud service communicatively and operatively coupled with system services, hardware resources, or software elements local to and/or external to the UPOE 325 host gaming headset 110. The depicted method 500 begins at step 505 with the processor 305 configuring physiological sensors in gaming headset 110 to capture physiological data from a user while the user plays a game. In various designs, the sensors may include EEG, HRV, or PPG physiological sensors and motion sensors, permitting the processor 305 to measure cognitive load, cognitive performance, and cognitive fatigue determined as a function of the sensor data. Then, the method continues at step 510 with the processor 305 capturing physiological data from the sensors while user plays the game. In some implementations the processor 305 may determine cognitive fatigue based on evaluating the measured cognitive load as a function of time. In various embodiments, the processor 305 may predict the user's risk of cognitive fatigue determined as a function of measured cognitive performance and a predictive analytic model trained based on reference cognitive performance data. The method continues at step 515 with the processor 305 measuring the user's cognitive performance and cognitive load determined as a function of the captured physiological data. The method continues at step 520 with the processor 305 predicting the user's cognitive error risk, cognitive fatigue and overall performance as a function of measured cognitive state and a machine learning model training on reference cognitive performance data. The method continues at step 525 with the processor 305 comparing, to a predetermined threshold, the predicted risk of poor cognitive performance or error, to determine if the user is at high risk of poor performance or an error playing the game, based on the comparison. The method continues at step 530 with the processor 305 performing a test to determine if the user's error risk, or poor performance risk, is high, based on the comparison performed by the processor 305 at step 525. Upon a determination by the processor 305 at step 530 the user's error risk, or poor performance risk, is high, the method continues at step 535 with the processor 305 notifying the user of the impending risk, and the method continues at step 510 with the processor 305 capturing physiological data from the sensors while the user plays the game. In various embodiments, the processor 305 may notify the user of the impending risk in various ways. For example, the processor 305 may notify the user of the impending risk by triggering an audibly or visibly detectable alert on the user's mobile device, gaming headset, or in the game the user is playing. Upon a determination by the processor 305 at step 530 the user's error risk, or poor performance risk, is not high, the method continues at step 510 with the processor 305 capturing physiological data from the sensors while the user plays the game. In various embodiments, the method may repeat.

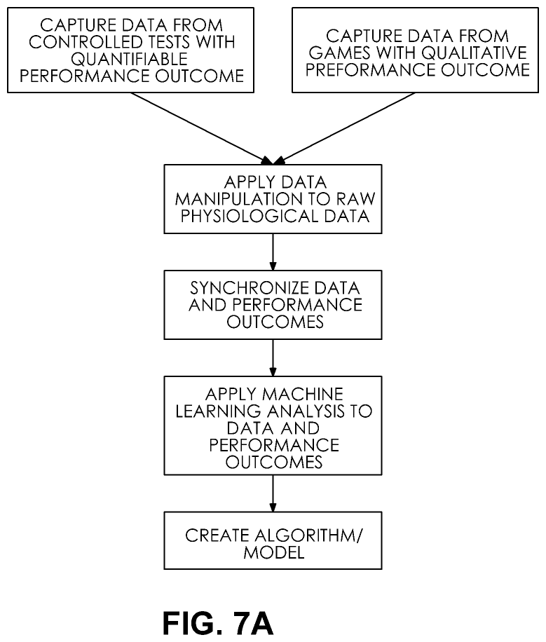

FIG. 6 depicts exemplary process steps to assess user performance according to a user mental function metric.

In one aspect, the user mental function metric may be cognitive fatigue. In another aspect, the user mental function metric may be error risk. In another aspect, the user mental function metric may be cognitive performance.

In FIG. 6, the depicted user performance assessment process steps include multiple stages configured in an exemplary sequence. In various examples, the depicted steps may be performed in any operable order. In the illustrated example, the user performance assessment process steps include capturing data from sensors, including, for example, EEG, PPG or ECG, in an illustrative first stage. In an illustrative second stage, the sensor data may be used to calculate measurements including brain wave patterns, or heart rate variability. In an illustrative third stage, the calculated measurements may be manipulated or processed to obtain higher-order characteristic data including, for example, ratios, rolling analysis, individualization, or equations. In an illustrative fourth stage, the higher-order characteristic data may be input to a predictive analytic model, such as, for example, a support vector machine, a neural network, a decision tree, an extreme gradient boosting model, or a random decision forest. In an illustrative example, the predictive analytic model may predict or measure a cognitive performance characteristic as a function of the ratios, rolling analysis, individualization, or equations, in an illustrative fifth stage. The cognitive performance characteristic may be based on one or more mental function metric, such as, for example: cognitive fatigue; error risk; cognitive performance; concentration or focus; stress; or, cognitive load or intensity. In an illustrative sixth stage, the predictive analytic output determined in the illustrative fifth stage may trigger a notification to a user, including, for example, an onboard audible or haptic alert, or a notification sent via a mobile or computer app.

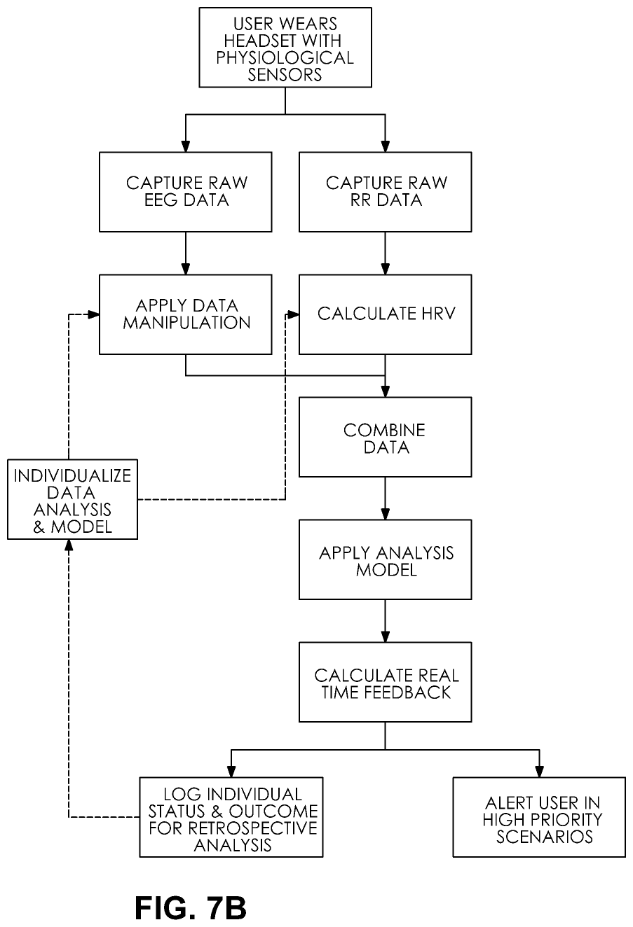

FIGS. 7A-7B together depict exemplary training and usage of an embodiment machine learning model configured to assess user performance according to a user mental function metric.

In one aspect, the user mental function metric may be cognitive fatigue. In another aspect, the user mental function metric may be error risk. In another aspect, the user mental function metric may be cognitive performance.

In FIG. 7A, the exemplary machine learning model is trained as a function of data fused from controlled tests with quantifiable performance outcomes, and from games with qualitative performance outcomes. Then, in the illustrated embodiment, data manipulation is applied to the raw physiological data. Then, in the depicted embodiment, the manipulated data is synchronized with performance outcomes. Then, in the illustrated embodiment, machine learning analysis is applied to the synchronized data and performance outcomes, and the machine learning model or algorithm is created.

In FIG. 7B, the exemplary trained machine learning model is applied to optimize gaming performance. In the illustrated embodiment, a user wears a headset configured with physiological sensors. Then, in the depicted example, raw EEG and RR data is captured. In the illustrated example, the EEG data is manipulated, and HRV is calculated from the raw RR data. Then, in the depicted embodiment, the manipulated EEG data, and calculated HRV are combined. Then, in the illustrated embodiment, real time feedback is calculated as a function of the combined data and the exemplary machine learning model trained as described with reference to FIG. 7A. In the depicted embodiment, the user is alerted in high priority scenarios, and individual status and outcome are logged for retrospective analysis. In the depicted embodiment, the machine learning model and performance data are individualized based on the logged status and outcome. In the depicted example, the subsequent EEG data manipulation and HRV calculation iterations are implemented as a function of the individualized machine learning model and performance data determined as a function of the individual status and outcome.

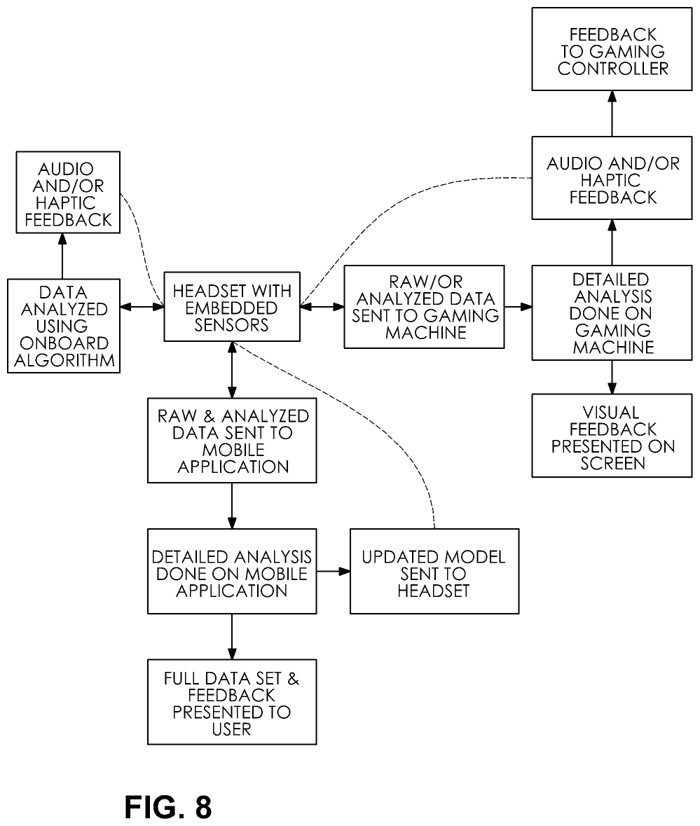

FIG. 8 depicts an exemplary information flow to assess user performance according to a user mental function metric.

In one aspect, the user mental function metric may be cognitive fatigue. In another aspect, the user mental function metric may be error risk. In another aspect, the user mental function metric may be cognitive performance.

In FIG. 8, the exemplary user performance assessment information flow begins with physiological data captured by a headset with embedded sensors. In some examples, the sensor data may be analyzed using an onboard algorithm, to provide audio and/or haptic feedback through the headset. Various embodiments may send raw and analyzed sensor data to a mobile application for detailed analysis, with the full data set and feedback presented to the user, and an updated model sent to the headset. Some embodiments may send raw and/or analyzed data to the gaming machine for detailed analysis, triggering visual in-game feedback presented on the gaming screen, with audio and/or haptic feedback through the gaming controller.

Although various embodiments have been described with reference to the Figures, other embodiments are possible. For example, some embodiments may measure cognitive load and cognitive fatigue determined as a function of physiological data detected by heart rate variability and EEG sensors mounted in headphones, headsets or head mounted units. In illustrative examples, cognitive load (or mental load) may be understood as the mental effort (intensity) used at the current moment to work on the provided task; cognitive fatigue (or mental fatigue) may be understood as the fatiguing impact of the cognitive load applied over time. Cognitive fatigue is a decrease in cognitive resources developing over time on sustained cognitive demands. In various implementations, a recovery recommendation may be provided based on the measurement of cognitive fatigue, in order to reduce fatigue. In an illustrative example, the recovery recommendation may include a relaxation schedule and/or monitored relaxation periods. Various embodiment designs may use EEG and HRV data to measure cognitive fatigue and provide real time feedback to the user.

Some embodiments may use physiological sensors as a means to quantify the level of cognitive fatigue measured via EEG, HRV and motion sensors embedded in a set of headphones/headset.

Various implementations may capture physiological measurements via sensors, and manipulate those measurements into meaningful information to quantify cognitive fatigue, and provide actionable feedback to the user in the fields of gaming, sports, information work, and physical work.

In an illustrative example, optimal cognitive performance cannot be achieved when excessive cognitive fatigue is present. Performing at a high level relies on a manageable level of fatigue. Performance can be reflected as an error to a challenge, a response time, quality of output, or volume of output.

High levels of cognitive fatigue can have a negative performance impact on activities such as computer games, sports, and many occupations including creative and development work.

Some embodiment designs may determine an individual's cognitive fatigue using physiological sensors to capture EEG, HRV and motion data from a head worn device, so that the user can view their level of cognitive fatigue, and be alerted to high levels that may result in reduced performance.

In an example illustrative of various embodiments' design and usage, EEG and HRV data is captured from the head, via sensors in a headset. The captured data is then manipulated, and individualized, before the system calculates a level of cognitive load for the user. This measure is then tracked via an accompanying mobile application, while high priority alerts may be given to the used via audio prompts in the accompanying headset. Feedback may also be accompanied by a specific recovery recommendation to assist a return to reduced fatigue and maximum performance as quickly as possible.

Some embodiments may use EEG, PPG and motion sensors embedded in a headset to capture EEG, HRV and movement data. This data is then manipulated and individualized to compare to the users historic profile to determine normal levels under various conditions. Personalized feedback is then provided to the user on their cognitive fatigue. This may be tracked via a mobile application, while audio prompts in the accompanying headset may alert the user when excessive levels of cognitive fatigue are evident.

In an illustrative example, actionable recommendations may accompany the cognitive fatigue measures. These recommendations may include practical steps, or suggestions, to aid the user to reduce fatigue and return to optimal function. One possible embodiment of this may include a recommended break duration from the current task, such as “A 1 hour break is recommended. Get away from screens, go for a walk, and resume in 1 hour to regain maximum performance.”

In an illustrative example, given the importance of personalized feedback, all measures are individualized based on the system's understanding of that user's normal profile. Various embodiments may start with a normative profile and adapt to the user's personal profile as the system gains data on the user from their use.

Various embodiment algorithms could be used to alert a user to the need for a recovery break, or change or task, when cognitive fatigue increases to a point where it limits the user's ability to perform well on their given task. The given task may be playing computer games, doing creative work, solving problems, or mental performance enhancement such as visualization. The recommendation of a break could be accompanied by a clear recommendation to assist the user to maximize the break so they can return to the task at full performance. In an illustrative example, the sensors to achieve this outcome are ideally embedded in a headset, such as a gaming headset, audio headphones, office communication headset, or even VR/AR.

Various embodiments may provide a head-worn system to monitor mental function and predict short term computer gaming performance, to provide real time feedback to the user, using physiological sensors which provide data for an analysis model that is updated based on the user's physiological response, and where possible, learned performance outcome.

Some embodiment design implementations may include a range of sensors, such as head mounted EEG, heart rate variability, and motion sensors, to monitor real time physiological measurements and activity of gamers. In some designs, an embodiment system may then give feedback to the gamer on the state of their current mental performance capability. An example may be, alerting the gamer to an increased risk of poor performance or an error, or providing an assessment of their response time or mental fatigue. In an illustrative example, feedback may be provided directly via the gaming machine, an accompanying mobile app, or directly via speakers accompanying the sensors (for example, headphones). Various embodiments may use EEG and HRV to optimize gaming performance. In some embodiments, EEG and HRV may be used to optimize gaming performance by providing real time feedback on cognitive fatigue and/or error risk.

An exemplary embodiment may include a sensor band in a gaming headset that captures EEG, heart rate variability and motion information, which is analyzed using a pre-trained model to provide real time feedback to the user on their cognitive fatigue, predicted response time, and overall cognitive performance, or error risk. The system may then use audible and/or haptic feedback via the headset to alert the user to a risk of poor performance, or error. As more physiological data is captured from the user, the model is personalized by learning normal response ranges for that individual, and further adapted when quantified performance measures are provided back to the system.

Illustrative feedback examples may include visual and/or haptic feedback from the gaming machine and accompanying controller, or feedback and notifications via a mobile app and accompanying smart watch app.

Various embodiments relate to use of physiological sensors as a means to quantify and enhance gaming performance. In an illustrative example, various designs may include the capture of physiological measurements via sensors during gaming, and the ability to manipulate those measurements into meaningful information to quantify gamer performance, and assist the gamer to improve their performance.

Some embodiments may provide a novel way of using the data from physiological sensors to aid gaming performance. In various embodiments, through the use of EEG, PPG and motion sensors, the system is able to measure and quantify cognitive performance. Cognitive performance includes, but is not limited to, cognitive fatigue, concentration, reaction time, and overall error risk.

Some embodiments may advantageously provide a prediction of cognitive fatigue determined as a function of captured sensor data. In various designs, a prediction of cognitive fatigue may be accompanied with a recommendation for a break if needed to recover, and thus sustain optimal performance.

Various embodiments may advantageously provide an error risk, that is, a risk of making a mistake and alert the user where an error is likely, determined as a function of captured sensor data.

In an illustrative example, although error risk may be influenced by cognitive fatigue, this is not the sole prediction. Rather, error risk is heavily impacted by the type of brain wave activity.

Various implementations may advantageously provide a performance score based on response time prediction determined as a function of captured sensor data, to give a user feedback on how well they are likely to perform, and accompanied by a recommendation of how to enhance their performance.

In various embodiments, EEG data may be used exclusively, or in conjunction with HRV and/or motion data, to assess performance state. In an illustrative example, feedback is then provided back to the gamer on their performance state. In some implementations, this can be done via an accompanying mobile application, via the headset itself (including audio or haptic via the communication headset), and/or directly via the game or gaming system.

Some embodiments may be configured with EEG, PPG and motion sensors embedded in a gaming headset to capture EEG, HRV and movement data. In some designs, these variables may then be individualized by comparing to the user's historic profile for these variables to determine normal levels under various conditions. In an illustrative example, some variable data may then be run through a machine learning model such as an extreme gradient boosting (XGB) model or random decision forest (RDF) to determine the user's short term risk of an error, or mistake. For example, when a high error risk is flagged, the system can make the user aware of the impending risk in an attempt to help them avoid the error. This feedback could be in the form of an audio prompt via the adjoining headset.

In an illustrative example, given the importance of individualizing some aspects of the data, in the case of a new user, a learning phase may be implemented whereby the system starts with normative values that are replaced with the user's personal data as the user uses the system more. This allows the system to deliver on the described experience initially, while becoming increasingly accurate for the individual user over time.

In some embodiments, the data captured from the sensors may be used in their raw format, or maybe analyzed in a variety of ways such as ratios, normalized against personal profile, equation, regression and machine learning models.

Various embodiments may use the same sensors to track the user's level of cognitive fatigue or load via an algorithm including individualized EEG and HRV data. In an illustrative example, this feedback is logged in a mobile application for detailed reporting, and feedback provided to the user when a recovery break is recommended due to excessive fatigue. The feedback maybe accompanied by a specific recommendation on how to optimize the break for maximum recovery, so gaming can be resumed at a high level.

Various embodiments may use the same sensors to track the user's level of cognitive fatigue or error risk via an algorithm including individualized EEG and HRV data. In an illustrative example, this feedback is logged in a mobile application for detailed reporting, and feedback provided to the user to reduce error risk.

Some embodiments may measure concentration or focus using sensor data. This measure may be tracked in a mobile app that is measured as an easy to interpret score out of 10 or 100.

Various designs may measure stress, using sensor data, and manipulating that data to achieve a stress score out of 10 or 100.

Some embodiments may include feedback to the user via visual stimuli, audio or haptic.

In an illustrative example, sensors may ideally be placed in the gaming headset, but may also be embedded in an independent head worn unit, or even in multiple locations such as a head unit for EEG and wrist worn unit for HRV.

In an illustrative example, performance in gaming is becoming increasingly popular with recreational gamers, and is the backbone of professional gamers being able to make money. The ability to monitor a gamer's cognitive performance will assist in guiding them to optimal gaming performance. Various embodiment algorithms may permit the determination of a gamer's error risk, cognitive fatigue, concentration and stress. These factors can, individually or in combination, allow feedback to the gamer to guide them to optimal performance. The sensors to achieve this outcome are ideally embedded in the gaming headset, or even VR/AR.

Various embodiments may advantageously permit recreational and professional gamers alike, to measure, quantify, track, and get actionable feedback on their cognitive readiness and performance, and how it relates to their gaming performance.

Some examples may improve a user's insight into how ready they may be to perform.

Such improved insight into performance readiness may be a result of more accurately predicting upcoming response times for the same task, based on using response time as a measure of performance. Various embodiments may advantageously provide a clear score and recommendation even when task-specific training is not possible or practical. This facilitation may be a result of a predicted performance score generated as a function of a response time prediction, and applying the performance score to subjective activities, or activities that may be similar, but not identical to an original training task. In an illustrative example, a gamer may put on their headset to play a game, and be informed with a performance score predicting how ready they are to perform, permitting them to select whether to compete, practice, or rest, based on the predicted performance score.

In various scenarios, poor performance as a result of cognitive fatigue may have negative consequences, including work safety, reduced productivity, and poorer performance in gaming. Using a combination of electroencephalogram (EEG) and, where applicable, heart rate variability (HRV), various embodiments identify when there is an increase in cognitive load and/or fatigue, providing the wearer with feedback and a recommendation to manage this fatigue. In an illustrative example, some embodiments may use EEG and HRV to provide real time feedback when the risk of an error/mistake is higher.

In various scenarios, errors may have negative consequences, including work safety, reduced productivity, and poorer performance in gaming. Using a combination of electroencephalogram (EEG) and, where applicable, heart rate variability (HRV), various embodiments identify when there is an increased risk of an error, providing the wearer with a warning of this increased risk in an attempt to avoid an error. In an illustrative example, some embodiments may use EEG and HRV to provide real time feedback when the risk of an error/mistake is higher.

Various embodiments may capture data from physiological sensors as a means to quantify the level of cognitive fatigue. Some embodiments may manipulate physiological measurements captured via sensors into meaningful information to quantify cognitive fatigue and error risk in a variety of fields including gaming, sports, information work, and physical work.

In an illustrative example, the performance of most mental and many physical activities are heavily reliant on cognitive decisions; whereby a better cognitive decision will have a positive impact on the outcome of the activity. Examples of such activities include computer games, sports, and many occupations including creative and development work. Participants of these activities have little, or no, knowledge of their current ability to make a good cognitive decision. This means they undertake the activity not knowing if they are about to perform at their best, or may be at increased risk of making mistakes and performing poorly. This lack of knowledge of their current ability to make a good cognitive decision may have a significant impact on their ability to achieve their goals. This highlights a need to be able to quantify cognitive fatigue in a variety of cognitive activities. Doing so will help the user avoid errors, optimize performance and achieve their goals.

In an illustrative example, the performance of most mental and many physical activities are heavily reliant on cognitive decisions; whereby a better cognitive decision will have a positive impact on the outcome of the activity. Examples of such activities include computer games, sports, and many occupations including creative and development work. Participants of these activities have little, or no, knowledge of their current ability to make a good cognitive decision. This means they undertake the activity not knowing if they are about to perform at their best, or may be at increased risk of making mistakes and performing poorly. This lack of knowledge of their current ability to make a good cognitive decision may have a significant impact on their ability to achieve their goals. This highlights a need to be able to quantify the risk of an error in a variety of cognitive activities. Doing so will help the user avoid errors, optimize performance and achieve their goals.

Some embodiments in accordance with the present disclosure may include determining when an individual is experiencing a high level of cognitive fatigue that will inversely impact the performance of their chosen activity. In an illustrative example, through the use of physiological sensors to capture EEG, HRV and motion data from a head worn device, the user can be notified when they are experiencing a high level of cognitive fatigue.

For example, in some embodiment implementations, EEG and HRV data may be captured from the head, with the data then manipulated, and in some cases, individualized, before being exposed to a machine learning model such as, for example, an extreme gradient boosting (XGB) model. In an illustrative example, the user may be warned when their cognitive fatigue is at a high level. This feedback may come in the form of an audio prompt via the accompanying headset, or via a mobile app.

Some embodiments in accordance with the present disclosure may include determining when an individual is experiencing a high level of error risk, or risk of performing poorly, that may inversely impact the performance of their chosen activity. In an illustrative example, through the use of physiological sensors to capture EEG, HRV and motion data from a head worn device, the user can be notified when they are experiencing a high level of error risk, or risk of performing poorly.

For example, in some embodiment implementations, EEG and HRV data may be captured from the head, with the data then manipulated, and in some cases, individualized, before being exposed to a machine learning model such as, for example, an extreme gradient boosting (XGB) model. In an illustrative example, the user may be warned when their error risk or risk of performing poorly is at a high level. This feedback may come in the form of an audio prompt via the accompanying headset, or via a mobile app.

In various examples of the present disclosure, the use of the word “mistake,” or “error,” is not isolated to an incorrect response to a challenge, but also may describe a poor performance in a challenge. An alternative definition for our use of these terms may include a slow response time to a challenge, or an undesirable outcome to a challenge. Some embodiments may use EEG, PPG and motion sensors embedded in a headset to capture EEG, HRV and movement data. Some of these variables are then individualized, by comparing the variables to the users historic profile for these variables, to determine normal levels under various conditions. Variable data may then be run through a machine learning model such as an extreme gradient boosting (XGB) model, which has learned from training data, to determine the user's short term risk of an error, or performing poorly. In some examples, when a high error risk is flagged, the system can make the user aware in an attempt to help them avoid the error. This feedback could be in the form of an audio prompt via the adjoining headset. In the case of a new user, a learning phase may be implemented whereby the system starts with normative values that are replaced with the user's personal data. This allows the system to deliver on the described experience initially, while becoming increasingly accurate for the individual user as they use the system over time. In some examples, sensors may be configured in a headset, but may also be embedded in an independent head worn unit or other wearable device, or even in multiple locations such as a head unit for EEG, and wrist worn unit for HRV. Some embodiments may include feedback to the user via visual stimuli, audio or haptic, via a headset, control unit, and/or app on a mobile device or computer. Various designs may be used by computer game players to receive alerts when they display high levels of cognitive fatigue, or used by knowledge workers who would be made aware when they are not performing at their best and likely to make suboptimal decisions. In an illustrative example, a worker undertaking a repetitive, or monitoring, task may be alerted by various designs to their high levels of cognitive fatigue, or not actioning important information. In various examples, data captured from the sensors maybe used in their raw format, or maybe analyzed in a variety of ways such as ratios, normalized against personal profile, equation, regression and machine learning models.

Some embodiment designs may include an algorithm to alert a user when they are displaying a high level of cognitive fatigue, allowing them to take steps to reduce this fatigue and avoid a negative impact on performance. Various application examples may include recreational activities such as gaming, and also work related activities. The sensors to achieve this outcome are ideally embedded in a headset, such as a gaming headset, audio headphones, office communication headset, or even VR/AR.

Some embodiment designs may include an algorithm to alert a user when they are at an increased short term risk of making an error, permitting the user to refocus, and perhaps avoid the impending error. Various application examples may include recreational activities such as gaming, and also work related activities. The sensors to achieve this outcome are ideally embedded in a headset, such as a gaming headset, audio headphones, office communication headset, or even VR/AR.

The use of in-home computer games entered popular culture in the 1980's and grew rapidly throughout the 1990's. During the early 2000's, the popularity of computer games continued to grow with the common use of online gaming.

Gaming has grown into a popular hobby for over 1 billion people, with serious gamers playing on PC and consoles, and online gaming accounting for an increasing portion of this user base. This is highlighted with recent data showing over 8 million users were playing Fortnite® online concurrently.

Gaming is no longer just a recreational activity. The introduction of tournaments have seen the introduction of professional gamers and organized professional teams. These events have grown to a scale where they have large live audiences, as well as viewer numbers of live streams that rival traditional sporting events viewership.

The competitive aspects of gaming extend to recreational gamers playing in their own homes. With a rapid increase in online gaming, inter-player competition has become a daily activity for millions of recreational players.

Both professional and recreational gamers are motivated to maximize their performance. However, it is not currently possible to know how ready the gamer is to perform. Cognitive variables such as fatigue and concentration have a large impact on the gamer's performance but, until now, have been unquantifiable.

In the Summary above and in this Detailed Description, and the Claims below, and in the accompanying drawings, reference is made to particular features of various embodiments of the invention. It is to be understood that the disclosure of embodiments of the invention in this specification is to be interpreted as including all possible combinations of such particular features. For example, where a particular feature is disclosed in the context of a particular aspect or embodiment of the invention, or a particular claim, that feature can also be used—to the extent possible—in combination with and/or in the context of other particular aspects and embodiments of the invention, and in the invention generally.

While multiple embodiments are disclosed, still other embodiments of the present invention will become apparent to those skilled in the art from this detailed description. The invention is capable of myriad modifications in various obvious aspects, all without departing from the spirit and scope of the present invention. Accordingly, the drawings and descriptions are to be regarded as illustrative in nature and not restrictive.

It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments.

In the present disclosure, various features may be described as being optional, for example, through the use of the verb “may;” or, through the use of any of the phrases: “in some embodiments,” “in some implementations,” “in some designs,” “in various embodiments,” “in various implementations,” “in various designs,” “in an illustrative example,” or “for example;” or, through the use of parentheses. For the sake of brevity and legibility, the present disclosure does not explicitly recite each and every permutation that may be obtained by choosing from the set of optional features. However, the present disclosure is to be interpreted as explicitly disclosing all such permutations. For example, a system described as having three optional features may be embodied in seven different ways, namely with just one of the three possible features, with any two of the three possible features or with all three of the three possible features.

In various embodiments. elements described herein as coupled or connected may have an effectual relationship realizable by a direct connection or indirectly with one or more other intervening elements.

In the present disclosure, the term “any” may be understood as designating any number of the respective elements, i.e. as designating one, at least one, at least two, each or all of the respective elements. Similarly, the term “any” may be understood as designating any collection(s) of the respective elements, i.e. as designating one or more collections of the respective elements, a collection comprising one, at least one, at least two, each or all of the respective elements. The respective collections need not comprise the same number of elements.

While various embodiments of the present invention have been disclosed and described in detail herein, it will be apparent to those skilled in the art that various changes may be made to the configuration, operation and form of the invention without departing from the spirit and scope thereof. In particular, it is noted that the respective features of embodiments of the invention, even those disclosed solely in combination with other features of embodiments of the invention, may be combined in any configuration excepting those readily apparent to the person skilled in the art as nonsensical. Likewise, use of the singular and plural is solely for the sake of illustration and is not to be interpreted as limiting.

In the present disclosure, all embodiments where “comprising” is used may have as alternatives “consisting essentially of,” or “consisting of” In the present disclosure, any method or apparatus embodiment may be devoid of one or more process steps or components. In the present disclosure, embodiments employing negative limitations are expressly disclosed and considered a part of this disclosure.

Certain terminology and derivations thereof may be used in the present disclosure for convenience in reference only and will not be limiting. For example, words such as “upward,” “downward,” “left,” and “right” would refer to directions in the drawings to which reference is made unless otherwise stated. Similarly, words such as “inward” and “outward” would refer to directions toward and away from, respectively, the geometric center of a device or area and designated parts thereof. References in the singular tense include the plural, and vice versa, unless otherwise noted.

The term “comprises” and grammatical equivalents thereof are used herein to mean that other components, ingredients, steps, among others, are optionally present. For example, an embodiment “comprising” (or “which comprises”) components A, B and C can consist of (i.e., contain only) components A, B and C, or can contain not only components A, B, and C but also contain one or more other components.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example, “at least 1” means 1 or more than 1. The term “at most” followed by a number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number),” this means a range whose limit is the second number. For example, 25 to 100 mm means a range whose lower limit is 25 mm and upper limit is 100 mm.

Many suitable methods and corresponding materials to make each of the individual parts of embodiment apparatus are known in the art. According to an embodiment of the present invention, one or more of the parts may be formed by machining, 3D printing (also known as “additive” manufacturing), CNC machined parts (also known as “subtractive” manufacturing), and injection molding, as will be apparent to a person of ordinary skill in the art. Metals, wood, thermoplastic and thermosetting polymers, resins and elastomers as may be described herein-above may be used. Many suitable materials are known and available and can be selected and mixed depending on desired strength and flexibility, preferred manufacturing method and particular use, as will be apparent to a person of ordinary skill in the art.

Any element in a claim herein that does not explicitly state “means for” performing a specified function, or “step for” performing a specific function, is not to be interpreted as a “means” or “step” clause as specified in 35 U.S.C. § 112 (f). Specifically, any use of “step of” in the claims herein is not intended to invoke the provisions of 35 U.S.C. § 112 (f). Elements recited in means-plus-function format are intended to be construed in accordance with 35 U.S.C. § 112 (f).

Recitation in a claim of the term “first” with respect to a feature or element does not necessarily imply the existence of a second or additional such feature or element.

The phrases “connected to,” “coupled to” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim in this or any application claiming priority to this application require more features than those expressly recited in that claim. Rather, as the following claims reflect, inventive aspects may lie in a combination of fewer than all features of any single foregoing disclosed embodiment. Thus, the claims following this Detailed Description are hereby expressly incorporated into this Detailed Description, with each claim standing on its own as a separate embodiment. This disclosure is to be interpreted as including all permutations of the independent claims with their dependent claims.

According to an embodiment of the present invention, the system and method may be accomplished through the use of one or more computing devices. As depicted, for example, at least in FIG. 1, FIG. 2, and FIG. 3, one of ordinary skill in the art would appreciate that an exemplary system appropriate for use with embodiments in accordance with the present application may generally include one or more of a Central processing Unit (CPU), Random Access Memory (RAM), a storage medium (e.g., hard disk drive, solid state drive, flash memory, cloud storage), an operating system (OS), one or more application software, a display element, one or more communications means, or one or more input/output devices/means. Examples of computing devices usable with embodiments of the present invention include, but are not limited to, proprietary computing devices, personal computers, mobile computing devices, tablet PCs, mini-PCs, servers or any combination thereof. The term computing device may also describe two or more computing devices communicatively linked in a manner as to distribute and share one or more resources, such as clustered computing devices and server banks/farms. One of ordinary skill in the art would understand that any number of computing devices could be used, and embodiments of the present invention are contemplated for use with any computing device.

In various embodiments, communications means, data store(s), processor(s), or memory may interact with other components on the computing device, in order to effect the provisioning and display of various functionalities associated with the system and method detailed herein. One of ordinary skill in the art would appreciate that there are numerous configurations that could be utilized with embodiments of the present invention, and embodiments of the present invention are contemplated for use with any appropriate configuration.

According to an embodiment of the present invention, the communications means of the system may be, for instance, any means for communicating data over one or more networks or to one or more peripheral devices attached to the system. Appropriate communications means may include, but are not limited to, circuitry and control systems for providing wireless connections, wired connections, cellular connections, data port connections, Bluetooth connections, or any combination thereof. One of ordinary skill in the art would appreciate that there are numerous communications means that may be utilized with embodiments of the present invention, and embodiments of the present invention are contemplated for use with any communications means.

Throughout this disclosure and elsewhere, block diagrams and flowchart illustrations depict methods, apparatuses (i.e., systems), and computer program products. Each element of the block diagrams and flowchart illustrations, as well as each respective combination of elements in the block diagrams and flowchart illustrations, illustrates a function of the methods, apparatuses, and computer program products. Any and all such functions (“depicted functions”) can be implemented by computer program instructions; by special-purpose, hardware-based computer systems; by combinations of special purpose hardware and computer instructions; by combinations of general purpose hardware and computer instructions; and so on—any and all of which may be generally referred to herein as a “circuit,” “module,” or “system.”

While the foregoing drawings and description may set forth functional aspects of the disclosed systems, no particular arrangement of software for implementing these functional aspects should be inferred from these descriptions unless explicitly stated or otherwise clear from the context.

Each element in flowchart illustrations may depict a step, or group of steps, of a computer-implemented method. Further, each step may contain one or more sub-steps. For the purpose of illustration, these steps (as well as any and all other steps identified and described above) are presented in order. It will be understood that an embodiment can contain an alternate order of the steps adapted to a particular application of a technique disclosed herein. All such variations and modifications are intended to fall within the scope of this disclosure. The depiction and description of steps in any particular order is not intended to exclude embodiments having the steps in a different order, unless required by a particular application, explicitly stated, or otherwise clear from the context.

Traditionally, a computer program consists of a sequence of computational instructions or program instructions. It will be appreciated that a programmable apparatus (i.e., computing device) can receive such a computer program and, by processing the computational instructions thereof, produce a further technical effect.

A programmable apparatus may include one or more microprocessors, microcontrollers, embedded microcontrollers, programmable digital signal processors, programmable devices, programmable gate arrays, programmable array logic, memory devices, application specific integrated circuits, or the like, which can be suitably employed or configured to process computer program instructions, execute computer logic, store computer data, and so on. Throughout this disclosure and elsewhere a computer can include any and all suitable combinations of at least one general purpose computer, special-purpose computer, programmable data processing apparatus, processor, processor architecture, and so on.

It will be understood that a computer can include a computer-readable storage medium and that this medium may be internal or external, removable and replaceable, or fixed. It will also be understood that a computer can include a Basic Input/Output System (BIOS), firmware, an operating system, a database, or the like that can include, interface with, or support the software and hardware described herein.

Embodiments of the system as described herein are not limited to applications involving conventional computer programs or programmable apparatuses that run them. It is contemplated, for example, that embodiments of the invention as claimed herein could include an optical computer, quantum computer, analog computer, or the like.

Regardless of the type of computer program or computer involved, a computer program can be loaded onto a computer to produce a particular machine that can perform any and all of the depicted functions. This particular machine provides a means for carrying out any and all of the depicted functions.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

Computer program instructions can be stored in a computer-readable memory capable of directing a computer or other programmable data processing apparatus to function in a particular manner. The instructions stored in the computer-readable memory constitute an article of manufacture including computer-readable instructions for implementing any and all of the depicted functions.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

The elements depicted in flowchart illustrations and block diagrams throughout the figures imply logical boundaries between the elements. However, according to software or hardware engineering practices, the depicted elements and the functions thereof may be implemented as parts of a monolithic software structure, as standalone software modules, or as modules that employ external routines, code, services, and so forth, or any combination of these. All such implementations are within the scope of the present disclosure.

Unless explicitly stated or otherwise clear from the context, the verbs “execute” and “process” are used interchangeably to indicate execute, process, interpret, compile, assemble, link, load, any and all combinations of the foregoing, or the like. Therefore, embodiments that execute or process computer program instructions, computer-executable code, or the like can suitably act upon the instructions or code in any and all of the ways just described.

The functions and operations presented herein are not inherently related to any particular computer or other apparatus. Various general-purpose systems may also be used with programs in accordance with the teachings herein, or it may prove convenient to construct more specialized apparatus to perform the required method steps. The required structure for a variety of these systems will be apparent to those of skill in the art, along with equivalent variations. In addition, embodiments of the invention are not described with reference to any particular programming language. It is appreciated that a variety of programming languages may be used to implement the present teachings as described herein, and any references to specific languages are provided for disclosure of enablement and best mode of embodiments of the invention. Embodiments of the invention are well suited to a wide variety of computer network systems over numerous topologies. Within this field, the configuration and management of large networks include storage devices and computers that are communicatively coupled to dissimilar computers and storage devices over a network, such as the Internet.

A number of implementations have been described. Nevertheless, it will be understood that various modifications may be made. For example, advantageous results may be achieved if the steps of the disclosed techniques were performed in a different sequence, or if components of the disclosed systems were combined in a different manner, or if the components were supplemented with other components. Accordingly, other implementations are contemplated within the scope of the following claims.

What is claimed is:

1. A computer-implemented process to assess task performance, the process comprising:
capturing physiological data from a sensor configured in a user's wearable device while the user performs a task;
individualizing the physiological data to the user based on comparison with historical user physiological data;
measuring the user's cognitive function determined as a function of the individualized physiological data; and,
automatically notifying the user of cognitive fatigue detected based on evaluating the measured cognitive load as a function of time.

2. The process of claim 1, wherein the wearable device further comprises a gaming headset.

3. The process of claim 1, wherein the task further comprises playing a game.

4. The process of claim 1, wherein the sensor further comprises an EEG sensor and the physiological data further comprises a signal encoding the user's brain activity.

5. The process of claim 1, wherein the physiological data further comprises HRV data encoding the user's cardiovascular activity.

6. The process of claim 1, wherein measuring the user's cognitive function further comprises evaluating the user's performance based on a mental function metric.

7. The process of claim 6, wherein the mental function metric further comprises power.

8. The process of claim 6, wherein the mental function metric further comprises pressure.

9. The process of claim 1, wherein notifying the user further comprises sending a notification from the user's wearable device to a mobile app configured in another device.

10. A computer-implemented process to assess gaming performance, the process comprising:
capturing live physiological data from a sensor configured in a user's gaming headset while the user plays a game, wherein the live physiological data comprises EEG and HRV data;
individualizing the live physiological data to the user based on comparison with historical user physiological data;
measuring the user's cognitive function determined as a function of: the individualized physiological data; a plurality of mental function metrics; and, a predictive analytic model trained with reference physiological data representative of a population of users playing a similar game; and,
automatically notifying the user of cognitive fatigue detected based on evaluating the measured cognitive function as a function of time.

11. The process of claim 10, wherein the live physiological data further comprises PPG data.

12. The process of claim 10, wherein the historical user physiological data further comprises data selected from the group consisting of EEG, HRV, and PPG.

13. The process of claim 10, wherein the plurality of mental function metrics further comprise power, and pressure.

14. The process of claim 10, wherein the predictive analytic model further comprises an RDF.

15. The process of claim 10, wherein measuring the user's cognitive function further comprises training an individualized predictive analytic model based on the individualized physiological data and the reference physiological data.

16. The process of claim 10, wherein notifying the user of cognitive fatigue further comprises triggering an indication visible to the user in the user's in-game field of view.

17. A computer-implemented process to assess gaming performance, the process comprising:
capturing live physiological data from a sensor configured in a user's gaming headset while the user plays a game, wherein the live physiological data comprises EEG, HRV, and PPG data;
individualizing the live physiological data to the user based on comparison with historical user physiological data, wherein the historical physiological data comprises EEG, HRV, and PPG data;
training an individualized predictive analytic model based on a baseline predictive analytic model, the individualized physiological data, and reference physiological data representative of a population of users playing a similar game;
measuring the user's cognitive load determined as a function of: the individualized physiological data; a plurality of mental function metrics; and, the individualized predictive analytic model; and,
automatically notifying the user of cognitive fatigue detected based on evaluating the measured cognitive load as a function of time.

18. The process of claim 17, wherein training the individualized predictive analytic model further comprises a controlled training technique.

19. The process of claim 17, wherein capturing live physiological data from the sensor further comprises artifact correction.

20. The process of claim 17, wherein the process further comprises a sensor location in accordance with the International 10-20 system.

21. A computer-implemented process to assess mental performance, the process comprising:
storing physiological data captured from a sensor configured in a user's wearable device during a task performance by the user;
determining if the task performance is complete;
in response to determining the task performance is complete:
individualizing the physiological data stored during the completed task performance to the user based on comparison with historical user physiological data;
measuring the user's cognitive function based on the individualized stored physiological data; and,
reporting the user's cognitive fatigue determined based on evaluating the measured cognitive load as a function of time.

22. The process of claim 21, wherein the task performance further comprises the user playing a game.

23. The process of claim 21, wherein reporting the user's cognitive fatigue further comprises providing the user feedback concerning the user's mental performance while the user performed the completed task.

24. The process of claim 23, wherein reporting the user's cognitive fatigue further comprises the user's mental performance evaluated as a function of the user's mental performance measured based on user performance of at least one task previous to the completed task.

25. The process of claim 21, wherein reporting the user's cognitive fatigue further comprises providing the user a prediction of the user's mental performance during a future task performance.
